(19) United States
(12) Patent Application Publication
(10) Pub. No.: US 2026/0263370 A1
(43) Pub. Date: Sep. 10, 2026

(54) DELIVERY OF PROTEIN EXTRACELLULARLY

(71) Applicants: Neil S. Forbes, Hadley, MA (US); Shradha Khanduja, Amherst, MA (US); Vishnu Raman, Sunderland, MA (US)

(72) Inventors: Neil S. Forbes, Hadley, MA (US); Shradha Khanduja, Amherst, MA (US); Vishnu Raman, Sunderland, MA (US)

(21) Appl. No.: 19/558,234

(22) Filed: Mar. 5, 2026

Related U.S. Application Data
(60) Provisional application No. 63/767,427, filed on Mar. 5, 2025.

(51) Int. Cl. A61K 9/50; A61K 38/21; A61P 35/00; C12N 1/20; C12N 9/36; C12N 15/70; C12N 15/74; C12R 1/19; C12R 1/42

(52) U.S. Cl. CPC A61K 9/5068; A61K 38/217; A61P 35/00; C12N 1/20; C12N 9/2462; C12N 15/70; C12N 15/74; C12R 2001/19; C12R 2001/42; C12Y 302/01017

(57) ABSTRACT

The described technology pertains to biotechnology, specifically engineered bacterial systems designed for extracellular delivery of therapeutic proteins in cancer treatment. The disclosed bacteria, such as Salmonella typhimurium, are genetically modified to include plasmid coding for a therapeutic protein, a salicylic acid-inducible promoter operably linked to a lysis protein, and a plasmid retention mechanism. These bacteria are modified to remain extracellular by incorporating knockouts such as flhD, which prevent intracellular invasion of cancer cells. Upon induction with salicylic acid, the bacteria lyse and release therapeutic proteins, such as IFNγ, into the tumor microenvironment, converting the environment from immunosuppressive to immunostimulatory and reducing tumor growth. The system is designed to provide safe and tumor-specific delivery, minimizing systemic toxicity. Applications include treating solid tumors, such as pancreatic and breast cancer.

### PRIORITY

This application claims the benefit of the filing date of U.S. provisional application Ser. No. 63/767,427, filed on Mar. 5, 2025, the disclosure of which is incorporated by reference herein.

### STATEMENT REGARDING FEDERALLY FUNDED RESEARCH

This invention was made with government support under grant number R21CA293765 awarded by the National Institutes of Health and grant number HT94252310067 awarded by the Department of Defense. The government has certain rights in this invention.

### INCORPORATION BY REFERENCE OF SEQUENCE LISTING

This application contains a Sequence Listing which has been submitted electronically in ST26 format and hereby incorporated by reference in its entirety. Said ST26 file, created on Feb. 27, 2026, is named 3724090US1.xml and is 73,728 bytes in size.

### FIELD OF INVENTION

The present disclosure pertains to biotechnology, specifically to engineered bacterial systems designed for extracellular delivery of therapeutic proteins in cancer treatment.

### BACKGROUND

Cell surface receptors bind extracellular growth factors and cytokines that are involved in multiple immune and growth regulation processes. In cancer, pro-inflammatory cytokines and growth factor receptor (GFR) antagonists can strongly reduce tumor burden by triggering an anti-tumor immune response or competitively inhibiting growth factor receptors thus, preventing tumor growth. However, intravenous delivery of anti-tumor cytokines and growth factor receptor antagonists can cause significant systemic toxicity.

### SUMMARY

According to an aspect of the present disclosure, a bacteria comprises a plasmid comprising a nucleic acid sequence coding for a therapeutic protein, a salicylic acid inducible promotor operably linked to a nucleic acid sequence coding for a lysis protein and a plasmid retention mechanism. According to some embodiments, the bacteria may not intracellularly invade cancer cells. According to some embodiments, the bacteria may be E. coli or Salmonella. According to some embodiments, the E. coli may be E. coli Nissle or MG1655. According to some embodiments, the Salmonella may be Salmonella typhimurium. According to some embodiments, the Salmonella may be non-motile.

According to some embodiments, the Salmonella may contain an flhD, fliC, flgE or an fliGHI knockout or any combination thereof. According to some embodiments, the salicylic acid inducible promoter may be from Acinetobacter baylyi.

According to some embodiments, the lysis protein may be lysin gene E from phage phiX174. According to some embodiments, the lysin gene E may contain a C-terminal ssrA degradation sequence. According to some embodiments, the C-terminal ssrA degradation sequence may comprise or consist of AANDENYALAA (SEQ ID NO: 53). According to some embodiments, the C-terminal ssrA degradation sequence may comprise or consist of AANDENYADAS (SEQ ID NO: 54). According to some embodiments, the C-terminal ssrA degradation sequence may comprise or consist of AANDENYNYADAS (SEQ ID NO: 55).

According to some embodiments, the therapeutic protein may be a globular protein. A globular protein is a type of protein that folds into a compact, roughly spherical shape, distinguishing it from fibrous proteins. These proteins are typically water-soluble due to hydrophilic amino acid residues on their surface and hydrophobic ones buried inside, examples include hemoglobin, myoglobin, albumin, trypsin, insulin, glucagon, immunoglobulins, carbonic anhydrase, DNA polymerase, ovalbumin, ferritin, IFN-gamma, type I interferons (e.g., IFN-alpha, IFN-beta), IL-10 family (e.g., IL-10, IL-19, IL-20, IL-22, IL-24, IL26), IL-12 and/or IL-23. According to some embodiments, the globular protein may be between 10 and 30 kilodaltons. According to some embodiments, the globular protein may be IFNγ.

According to some embodiments, the plasmid retention mechanism may be a genomic asd knockout combined with pAsd-asd expressed on the plasmid.

According to an aspect of the present disclosure, a method to treat cancer comprises administering the bacteria comprising a plasmid comprising a nucleic acid sequence coding for a therapeutic protein, a salicylic acid inducible promotor operably linked to a nucleic acid sequence coding for a lysis protein and a plasmid retention mechanism to subject in need thereof. According to some embodiments, the subject may be human. According to some embodiments, the cancer may comprise a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma or retinoblastoma.

These and other features will be more clearly understood from the following description taken in conjunction with the accompanying drawings and claims.

### DRAWINGS

The drawings illustrate generally, by way of example, but not by way of limitation, various embodiments discussed herein.

FIG. 1. ED Sal-IFNγ when injected intravenously colonizes tumors, lyses extracellularly and release IFNγ. After an intravenous injection of ED Sal-IFNγ, it colonizes tumors, clears from healthy organs. After induction with, for example, salicylic acid, it lyses extracellularly and delivers IFNγ into tumor microenvironment. This release converts the tumor microenvironment from immunosuppressive to immunostimulatory and causes reduction in tumor volumes.

FIGS. 2A-2C. flhDC-Salmonella are engineered to not invade cancer cells. A) Salmonella was engineered to be non-invasive and deliver extracellularly by deletion of flhD and the addition of a salicylic acid inducible lysis circuit. B) Parental Salmonella were abundantly found inside cancer cells (green) while the flhD-strain was minimally invasive C) While the parental strain invaded over 80% of cancer cells, ED Sal only invaded 10% of cancer cells (****, P<0.0001).

FIGS. 3A-3G: Tuning lysin E protein degradation enables bacterial lysis. A) Lysin gene E was placed under control of the salicylic acid inducible promoter. B) A C-terminal ssrA peptide sequence (Slow, medium or fast degradation) was added in order to accelerate proteasomal degradation, thereby tuning lysin E expression levels. C) In the absence of salicylic acid, all three bacterial strains grew in luria broth. However, Salmonella containing the slow degradation grew slower than either the Salmonella expressing the lysin E-ssrA (medium) or lysin E-ssrA (fast) degradation tags (***, P<0.001; ****, P<0.0001). D) After 500 μM salicylic acid induction, only Salmonella expressing the lysin E-ssrA (medium) gene reduced in concentration, indicating bacterial lysis, while both the slow and fast strains continued to grow (**, P<0.01; ***, P<0.001). Salmonella expressing lysin E with either the (E) fast or (G) slow ssrA degradation tags did not reduce in culture density, indicating minimal bacterial lysis, as compared to Salmonella expressing lysin E with a (F) medium strength ssrA degradation tag.

FIGS. 4A-4D: A physiological concentration of Salicylic acid triggers ED Sal lysis and protein release. A) A concentration of 500 nM of Salicylic acid was enough to induce significant bacterial lysis and decrease the growth rate of ED Sal. B) At concentrations of 500 nM or higher, 99.99% of ED Sal lysed and C) released greater than 140 μg/ml of IFN gamma (****, P<0.0001). D) Full length IFN gamma was released after 500 μM Salicylic acid induction (red arrow, 19 kDa).

FIGS. 5A-5F. ED Sal released IFNγ induces MHC I upregulation on B16F10 cancer cells and macrophage polarization in vitro. A) Salmonella expressing IFNγ are induced with salicylic acid and lyse, releasing the cytokine into the surrounding environment. The cytokine binds with cancer cell IFNγ receptor and triggers a signaling cascade that upregulates MHC I expression. B) MHC I upregulation steadily increased at concentrations ranging from 500 nM to 100 μM but decreased at 500 μM. C) The MHC I expression level of B16F10 cancer cells peaked at concentrations of Salicylic acid induction between 10 and 100 μM (*, P<0.05; **, P<0.01). D) ED Sal-IFNγ lyse and polarize macrophages towards an M1 phenotype in vitro. E) Salicylic acid induction concentrations from 500 nM to at least 500 μM significantly increased RAW macrophage polarization. F) Salicylic acid induction increased CD80 expression on macrophages significantly more than uninduced and ED Sal-GFP (*, P<0.05; **, P<0.01, ****, P<0.0001).

FIGS. 6A-6D. Systemic delivery of IFN gamma with ED Sal reduces pancreatic cancer growth in vivo. A) Mice with subcutaneous KPCY tumor cells were intravenously injected with ED Salmonella. 48 hours after bacterial injection, 50 mg/ml of salicylic acid was intraperitoneally administered to the mice to induce bacterial lysis and IFN gamma delivery within tumors. Bacterial administration was repeated two more times and tumors were measured every three days. B) ED Sal-IFN gamma significantly reduced tumor growth over the course of 6 days as compared to the control, ED Sal-GFP. C) Delivery of IFN gamma with ED Sal reduced tumor volumes by 40% as compared to the control (*, P<0.05). D) 100% of ED Sal-IFN gamma treated mice survived longer than the ED Sal-GFP treated mice (*, P<0.01).

FIGS. 7A-7H. Systemic delivery of IFN gamma with ED Sal and spike reduces breast cancer growth in vivo. A) Mice with subcutaneous E0771 tumor cells were intravenously injected with ED Salmonella and bacteria transformed with a spike plasmid. 48 hours after bacterial injection, 50 mg/ml of Salicylic acid was intraperitoneally administered to the mice to induce bacterial lysis and IFN gamma delivery within tumors. Bacterial administration was repeated two more times and tumors were measured every three days. B) ED Sal-IFN gamma+Spike significantly reduced tumor growth over the course of 6 days as compared to the control, ED Sal-GFP+Spike. C) Delivery of IFN gamma with ED Sal and Spike reduced tumor volumes by 60% as compared to the control (**, P<0.01). D) 100% of ED Sal-IFN gamma treated mice survived longer than the ED Sal-GFP treated mice (*, P<0.01). E-H) Tumors treated with Sal-IFN had higher numbers of E) infiltrated CD3 T cells (**, P=0.0098), F) CD8+ T cells (*, P=0.0109), G), CD80+ activated macrophages (**, P=0.0010), and H) CD11c+ dendritic cells (*, P=0.0486).

FIGS. 8A-8B. Salicylic acid induction triggers extracellular lysis of ED Sal-IFNγ and rapidly reduces tumor bacterial burden. (A) C57BL/6 mice bearing subcutaneous KPCY tumors were injected intravenously on Day 0 with 1*10⁷ CFU of ED Sal-IFNγ. On Day 3, one cohort received an intraperitoneal injection of salicylic acid to activate the lysis circuit, while the control cohort remained uninduced. All mice were euthanized 24 hours later, and major organs including liver, spleen, kidneys, lungs, heart, and tumor were harvested for bacterial quantification. (B) Bacterial density (log CFU/g) across organs in uninduced and salicylic acid induced mice. Induction caused modest reductions in bacterial burden in healthy organs (1.5- to 5.5-fold decreases). In tumors, salicylic acid induction produced a pronounced 17-fold reduction in viable bacterial density compared with uninduced controls (*, P=0.030). Data are shown as means±SEM. The statistical comparison in (B) are Student's t-tests with Welch's correction. Asterisks indicate significance (*, P<0.05).

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Delivering highly potent, extracellular therapies with a tumor-specific delivery vehicle maximizes tumor efficacy while simultaneously preventing systemic cytotoxicity. Provided herein is a clinically translatable bacterial-based, such as Salmonella-based, extracellular delivery platform.

Traditional extracellularly active cancer therapies, like antibodies, cytokines and competitive inhibitors require upregulation of specific receptors on tumor cells in order to be efficacious. However, receptors that are overexpressed within tumor tissue are also expressed within healthy organ tissue. Systemically delivered therapies can also be rapidly cleared or sequestered in healthy organ tissue, thus preventing effective tumor accumulation. As a result, systemically administered antibody and cytokine therapies can fail to demonstrate efficacy because of significant toxicity and/or ineffective tumor accumulation.

Salmonella, and other specific bacteria, specifically accumulate in tumors at ratios of 10,000:1 over any other organ in the body, grow exponentially only within tumors and rapidly clear from healthy organ tissue. As a result, the bacteria are capable of delivering 1,000-10,000 times more therapy in tumors compared to the initially administered dose. As a result, Salmonella-mediated delivery of antibody and cytokine-based therapies can maximize tumor efficacy while minimizing systemic toxicity. Moreover, Salmonella can deliver a wide range of non-specific, cytotoxic and immunotherapeutic biologicals by virtue of the bacteria's highly focused tumor accumulation.

Provided herein is a safe, clinically applicable, tumor-specific, bacterial, such as Salmonella-based, extracellular delivery vehicle that is different in at least two ways: 1) the bacterial system is not responsive to arabinose. Instead, it only requires an over-the-counter dose of aspirin to deliver therapies extracellularly within tumors and 2) instead of secreting the therapy, the bacteria lyse in the presence of OTC aspirin and therefore, can deliver a much wider range of therapies that bacteria are able to produce but unable to secrete.

While extracellular delivery vehicles have been created using E. coli and Salmonella, they either have utilized 1) an arabinose inducible delivery system or autonomous, quorum sensing based delivery, 2) ultra-sound-based induction and/or 3) secretion system.

Moreover, to create this extracellular lysis system, the expression of the lysis protein, lysin gene E, was tuned using a ssrA protein degradation system. A ssrA protein degradation system is a mechanism used to regulate protein stability and degradation within cells. It involves tagging proteins with a specific peptide sequence that signals for their degradation by the cell's proteolytic machinery. This system is used in synthetic biology and genetic engineering to control protein levels within a cell. A ssrA protein degradation system can include: an ssrA Tag (the ssrA tag is a short peptide sequence that is added to the C-terminus of a protein. This tag serves as a signal for the protein to be recognized and degraded by the cell's protease systems, such as the ClpXP or ClpAP protease complexes); degradation rate (the rate at which a protein is degraded can be tuned by modifying the sequence of the ssrA tag. Different sequences can lead to different degradation rates, allowing for precise control over protein stability and abundance; and applications (such as ssrA protein degradation system to fine-tune the expression levels of the lysis protein, e.g., lysin gene E. By adjusting the degradation rate of lysin E, the system ensures that the protein is expressed at optimal levels to induce bacterial lysis in response to salicylic acid, without causing toxicity to the bacteria in the absence of the inducer). Overall, the ssrA protein degradation system provides a versatile tool for controlling protein levels in engineered organisms, enabling precise regulation of cellular processes.

Engineering lysin E gene expression using this C-terminal ssrA protein degradation system was needed to create a functional bacterial delivery system. A high level of lysis gene expression, such as lysin E expression, resulted in toxicity to the bacteria in the absence of, for example, salicylic acid. The bacteria evolved resistance and were resistant to lysis and extracellular therapeutic delivery when lysin E expression was too high. Conversely, when lysin E expression was too low, the bacteria did not lyse because too little of the protein was expressed. As a result, lysin E expression needed to be fine-tuned in order to be responsive to physiological concentrations of salicylic acid. This expression tuning system of lysin gene E is unique to this strain and has never been used by other researchers for the purposes of tumor-specific, extracellular protein delivery.

In addition, a strain of Salmonella had to be developed that safely colonized tumors while remaining extracellular. To achieve this goal, flagella expression was removed and a sseJ knockout was implemented to attenuate the strain to improve its safety. These knockouts enabled the bacteria to grow and remain extracellular in tumors.

```
SseJ (accession no. CBW17656.1):
(SEQ ID NO: 1)
    1 MPLSVGQGYF TSSISSEKEN AIKESARLPE LSLWEKIKAY FFTTHHAEAL ECIFNLYHHQ
  61 ELNLTPVQVR GAYIKLRALA SQGCKEQFII ESQEHADKLI IKDDNGENIL SIEVECHPEA
121 FGLAKEINKS HPKPKNISLG DITRLVFFGD SLSDSLGRMF EKTHHILPSY GQYFGGRFTN
181 GFTWTEFLSS PHELGKEMLN FAEGGSTSAS YSCENCIGDF VSNTDRQVAS YTPSHQDLAI
241 FLLGANDYMT LHKDNVIMVV EQQIDDIEKI ISGGVNNVLV MGIPDLSLTP YGKHSDEKRK
301 LKDESIAHNA LLKTNVEELK EKYPQHKICY YETADAFKVI MEAASNIGYD TENPYTHHGY
361 VHVPGAKDPQ LDICPQYVEN DLVHPTQEVH HCFAIMLESF
or at least 90%, 95%, 96%, 97%, 98%, 99% or 100%
identity thereto.
sseJ sequence (DNA) - Accession number-NCBI Reference Sequence: NC_016856.1
(SEQ ID NO: 2)
ATGCCATTGAGTGTTGGACAGGGTTATTTCACATCATCTATCAGTTCTGAAAAATTTAATGCGATAAAAGAAAGCGC
ACGCCTTCCGGAATTAAGTTTATGGGAGAAAATCAAAGCATATTTCTTTACCACCCACCATGCAGAGGCGCTCGAAT
GTATCTTTAATCTTTACCACCATCAGGAACTGAATCTAACACCGGTACAGGTTCGCGGAGCCTACATCAAACTTCGA
GCCTTAGCGTCTCAGGGATGTAAAGAACAGTTTATTATAGAATCACAGGAACACGCCGATAAGTTGATTATTAAAGA
TGATAATGGTGAAAATATTTTGTCTATTGAGGTTGAATGTCATCCGGAAGCTTTTGGTCTTGCAAAAGAAATCAATA
AATCACATCCCAAGCCCAAAAATATTTCTTTGGGTGATATTACCAGACTGGTATTTTTTGGCGACAGCTTGTCTGAC
TCCTTAGGGCGTATGTTTGAAAAAACACATCATATCTTACCCTCCTATGGTCAATACTTTGGCGGAAGGTTTACTAA
TGGATTTACCTGGACTGAGTTTTTATCATCTCCACACTTCTTAGGTAAAGAGATGCTTAATTTTGCTGAAGGGGGAA
GTACATCGGCAAGCTATTCCTGCTTTAATTGCATCGGTGACTTTGTATCAAATACGGACAGACAAGTCGCATCTTAC
ACCCCTTCTCACCAGGACCTGGCGATATTTTTATTGGGGGCTAATGACTATATGACACTACACAAAGATAATGTAAT
AATGGTCGTTGAGCAACAAATTGATGATATTGAAAAAATAATTTCCGGTGGAGTTAATAATGTTCTGGTCATGGGGA
TTCCCGATTTGTCTTTAACACCTTATGGCAAACATTCTGATGAAAAAAGAAAGCTTAAGGATGAAAGCATCGCTCAC
AATGCCCTGTTAAAAACTAATGTTGAAGAATTAAAAGAAAAATACCCCCAGCATAAAATATGCTATTACGAGACTGC
CGATGCATTTAAGGTGATAATGGAGGCGGCCAGTAATATTGGTTATGATACGGAAAACCCTTATACTCACCACGGCT
ATGTACATGTTCCCGGGGCTAAAGACCCTCAGCTAGATATATGTCCGCAATACGTCTTCAACGACCTTGTCCATCCA
ACCCAGGAAGTCCATCATTGTTTTGCCATAATGTTAGAAAGTTTTATAGCTCATCATTATTCCACTGAATAA
or at least 90%, 95%, 96%, 97%, 98%, 99% or 100% identity thereto.
```

In addition, the aspirin inducible system enables extracellular therapeutic delivery and facilitates bacterial clearance after delivery, thus, significantly improving safety.

To demonstrate efficacy, IFN-gamma was delivered selectively within pancreatic tumors. IFN-gamma is a highly potent anti-tumor cytokine that generates an anti-tumor immune response. However, systemic delivery of IFN-gamma causes significant toxicity and IFN-gamma has a short (30 minute) half-life in circulation. As a result, IFN-gamma was the ideal cytokine to deliver to demonstrate proof-of-concept of safety and efficacy of the extracellular delivery strain of Salmonella.

Thus, provided herein is a tumor-specific, extracellular protein delivery method and compositions. This invention improves the efficacy of several cancer therapies while simultaneously improving quality-of-life of cancer patients. Since this strain of Salmonella selectively colonizes and delivers protein into the tumor microenvironment, it maximizes anti-tumor efficacy without creating systemic toxicity.

There is nothing presently on the market that delivers protein therapies extracellularly in a highly tumor specific manner. An autonomous quorum sensing lysis system has been used to deliver extracellular therapies using E. coli Nissle (Danino). However, this system depends on a high density of bacteria specifically within the tumor to trigger autonomous delivery. The system provided herein delivers into tumors independent of bacterial tumor density as it only requires an over-the-counter dose of aspirin.

The Salmonella described herein deliver 3+ orders of magnitude more therapy within the tumor microenvironment compared to the initially administered dose. This is because of the unique ability of Salmonella to grow exponentially within tumors while rapidly clearing from healthy organ tissue. Moreover, the ability to control delivery using an over-the-counter dose of aspirin makes this approach highly controllable and patient friendly.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 16th Edition, by M. Larranga, R Lewis Sr., and R Lewis, New York, N.Y., 2016.

For the purposes of clarity and a concise description, features can be described herein as part of the same or separate embodiments; however, it will be appreciated that the scope of the invention may include embodiments having combinations of all or some of the features described.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described. As used herein, the term “in some embodiments” refers to embodiments of all aspects of the disclosure, unless the context clearly indicates otherwise.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and PASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, an “effective amount” or “therapeutically effective amount” (terms can be used interchangeably) means an amount sufficient to produce a selected effect, such as alleviating symptoms of a disease or disorder. A “therapeutically effective amount” of a treatment is a predetermined amount calculated to achieve the desired effect, i.e., to treat, combat, ameliorate, prevent, or improve one or more symptoms of, for example, a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the circumstances surrounding the case, including, for example, the compound/composition/agent/fusion protein administered, the route of administration, and the condition being treated or prevented. It will be understood that the effective amount administered will be determined by the physician in the light of the relevant circumstances including the condition to be treated, the choice of compound to be administered, and the chosen route of administration, and therefore any dosage ranges provided herein are not intended to limit the scope of the present disclosure in any way.

The term “delivery vehicle” or “carrier” refers to any kind of device or material which can be used to deliver the invention in vivo.

The term “treat,” “treated,” or “treating” refers to therapeutic treatment and/or prophylactic or preventative measures wherein the object is to prevent or slow down (lessen) an undesired physiological condition, disorder, or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, the term “therapeutic” means an agent utilized to treat, combat, ameliorate, prevent, or improve an unwanted condition or disease of a patient.

The term “in need thereof” means that the subject has been identified or suspected as having a need for the particular method or treatment. In some embodiments, the identification can be by any means of diagnosis or observation. In any of the methods described herein, the subject can be in need thereof.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, ophthalmic, or pulmonary.

As used herein, a “subject in need thereof” is a patient, animal (domestic (cat, dog) or farm animal (livestock, horse, cow), mammal, or human, who will benefit from the method of this invention. The terms “subject” refers to an animal, such as a mammalian species (e.g., human). More specifically, a subject can be a vertebrate, e.g., a mammal such as a mouse, a primate, a simian, or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that needs therapy or suspected of needing therapy. The terms “individual” or “patient” are intended to be interchangeable with “subject.”

A disease, condition, or disorder is “alleviated” if the severity of a symptom of the disease or disorder, the frequency with which such a symptom is experienced by a subject, or both, are reduced.

The terms “inhibit”, “inhibiting”, and “inhibition” refer to the slowing, halting, or reversing the growth or progression of a disease, condition, group of cells, protein or its expression. The inhibition can be greater than about 20%, 40%, 60%, 80%, 90%, 95%, or 99%, for example, compared to the growth or progression that occurs in the absence of the treatment or contacting.

“Expression” refers to the production of RNA from DNA and/or the production of protein directed by genetic material (e.g., RNA (mRNA)). Inducible expression, as opposed to constitutive expression (expressed all the time), is expression which only occurs under certain conditions, such as in the presence of specific molecule (e.g., arabinose) or an environmental que.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a nonnaturally-occurring nucleic acid is considered to be exogenous to a host once in the host. It is important to note that non-naturally occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally occurring nucleic acid. It follows that genomic DNA fragments produced by PCR or restriction endonuclease treatment as well as cDNAs are considered to be non-naturally occurring nucleic acid since they exist as separate molecules not found in nature. An exogenous sequence may therefore be integrated into the genome of the host. It also follows that any nucleic acid containing a promoter sequence and polypeptide-encoding sequence (e.g., cDNA or genomic DNA) in an arrangement not found in nature is non-naturally occurring nucleic acid. A nucleic acid that is naturally occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

Flagella are filamentous protein structures found in bacteria, archaea, and eukaryotes, though they are most commonly found in bacteria. They are typically used to propel a cell through liquid (i.e., bacteria and sperm). However, flagella have many other specialized functions. Flagella are usually found in gram-negative bacilli. Gram-positive rods (e.g., Listeria species) and cocci (some Enterococcus species, Vagococcus species) also have flagella.

Engineered Salmonella could be any strain of Salmonella designed to lyse and deliver protein intracellularly.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A: T and G: C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and including at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC 5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G: C ratio within the nucleic acids.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e., analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence, e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “kit” refers to a combination of reagents and/or apparatus for administering the compositions provide herein. In some embodiments, a kit may further include one or more apparatus to facilitate sample harvesting. In some embodiments, a kit may further include one or more reagents for sample processing. In some embodiment s, a kit may further include one or more written instructions.

The term “sample” is used herein in the broadest sense and can be obtained from any source in the body. A sample can encompass fluids, solids and/or tissues. In some embodiments, a sample can include one or more of the following fluids: oral fluid, nasal fluid, or ear drainage. A sample can also include other fluids, such as serous fluid, urine, saliva, tears, blood, plasma, and serum. In some embodiments, the sample is an oral fluid, nasal fluid, saliva, blood, plasma, or serum.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

Methods involving conventional molecular biology techniques are described herein. Such techniques are generally known in the art and are described in detail in methodology treatises, such as Molecular Cloning: A Laboratory Manual, 4th ed., Green and Sambrook, Cold Spring Harbor Laboratory Press, Cold Spring Harbor, N.Y., 2014; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

### I. Bacteria/Flagella

Bacteria useful in the invention include, but are not limited to, Clostridium, Bifidus, Escherichia coli or Salmonella, T3SS-dependent bacteria, such as shigella, salmonella and Yersinia Pestis. Further, E. coli can be used if the T3SS system is place in E. coli.

### Salmonella

Examples of Salmonella strains which can be employed in the present invention include, but are not limited to, Salmonella typhi (ATCC No. 7251) and S. typhimurium (ATCC No. 13311). Attenuated Salmonella strains include S. typhi-aroC-aroD (Hone et al. Vacc. 9:810 (1991) S. typhimurium-aroA mutant (Mastroeni et al. Micro. Pathol. 13:477 (1992)) and Salmonella typhimurium 7207. Additional attenuated Salmonella strains that can be used in the invention include one or more other attenuating mutations such as (i) auxotrophic mutations, such as aro (Hoiseth et al. Nature, 291:238-239 (1981)), gua (McFarland et al Microbiol. Path., 3:129-141 (1987)), nad (Park et al. J. Bact, 170:3725-3730 (1988), thy (Nnalue et al. Infect. Immun., 55:955-962 (1987)), and asd (Curtiss, supra) mutations; (ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop.sup.c (Miller et al. J. Bact, 172:2485-2490 (1990)) or ompR (Dorman et al. Infect. Immun., 57:2136-2140 (1989)) mutations; (iii) mutations that modify the stress response, such as recA (Buchmeier et al. Mol. Micro., 7:933-936 (1993)), htrA (Johnson et al. Mol. Micro., 5:401-407 (1991)), htpR (Neidhardt et al. Biochem. Biophys. Res. Com., 100:894-900 (1981)), hsp (Neidhardt et al. Ann. Rev. Genet, 18:295-329 (1984)) and groEL (Buchmeier et al. Sci., 248:730-732 (1990)) mutations; mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d'Hauteville et al. Mol. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 (1993)) mutations; (v) mutations that affect DNA topology, such as top A (Galan et al. Infect. Immun., 58:1879-1885 (1990)); (vi) mutations that disrupt or modify the cell cycle, such as min (de Boer et al. Cell, 56:641-649 (1989)); (vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phIA (Molin et al. Ann. Rev. Microbiol., 47:139-166 (1993)); (viii) mutations that alter the biogenesis of lipopolysaccharide and/or lipid A, such as rFb (Raetz in Escherichia coli and Salmonella typhimurium, Neidhardt et al, Ed., ASM Press, Washington D.C. pp 1035-1063 (1996)), galE (Hone et al. J. Infect. Dis., 156:164-167 (1987)) and htrB (Raetz, supra), msbB (Reatz, supra; and U.S. Pat. No. 7,514,089); and (ix) introduction of a bacteriophage lysis system, such as lysogens encoded by P22 (Rennell et al. Virol, 143:280-289 (1985)), lamda murein transglycosylase (Bienkowska-Szewczyk et al. Mol. Gen. Genet., 184:111-114 (1981)) or S-gene (Reader et al. Virol, 43:623-628 (1971)).

The attenuating mutations can be either constitutively expressed or under the control of inducible promoters, such as the temperature sensitive heat shock family of promoters (Neidhardt et al. supra), or the anaerobically induced nirB promoter (Harbome et al. Mol. Micro., 6:2805-2813 (1992)) or repressible promoters, such as uapA (Gorfinkiel et al. J. Biol. Chem., 268:23376-23381 (1993)) or gcv (Stauffer et al. J. Bact, 176:6159-6164 (1994)).

In one embodiment, the bacterial delivery system is safe and based on a non-toxic, attenuated Salmonella strain that has a partial deletion of the msbB gene. This deletion diminishes the TNF immune response to bacterial lipopolysaccharides and prevents septic shock. In another embodiment, it also has a partial deletion of the purI gene. This deletion makes the bacteria dependent on external sources of purines and speeds clearance from non-cancerous tissues (13). In mice, the virulence (LD₅₀) of the therapeutic strain is 10,000-fold less than wild-type Salmonella (72, 73). In pre-clinical trials, attenuated Salmonella has been administered systemically into mice and dogs without toxic side effects (17, 27). Two FDA-approved phase I clinical trials have been performed and showed that this therapeutic strain can be safely administered to patients (20). In one embodiment, the strain of bacteria is VNP20009, a derivative strain of Salmonella typhimurium. Deletion of two of its genes-msbB and purI-resulted in its complete attenuation (by preventing toxic shock in animal hosts) and dependence on external sources of purine for survival. This dependence renders the organism incapable of replicating in normal tissue such as the liver or spleen, but still capable of growing in tumors where purine is available.

Further, insertion of a failsafe circuit into the bacterial vector prevents unwanted infection and defines the end of therapy without the need for antibiotics to remove the bacteria (e.g., salmonella).

### Flagella

Provided herein is the addition or deletion of flagellar sequences from the bacteria.

1) flhDC Sequence

In one aspect, the flhDC sequence is the bicistronic, flhDC coding region found in the Salmonella typhimurium 14028s strain or a derivative thereof

### Accession Number—

fhD-NCBI Reference Sequence: NC_016856.1
flhC-NCBI Reference Sequence: NC_016856.1

Bicistronic DNA sequence
(SEQ ID NO: 3)
ATGCATACATCCGAGTTGCTAAAACACATTTATGACATCAATTTGTCATATTTACTCCTT
GCACAGCGTTTGATCGTCCAGGACAAAGCATCTGCGATGTTCCGCCTCGGTATCAACG
AAGAGATGGCAAACACACTGGGCGCGTTGACCCTGCCGCAGATGGTCAAACTGGCG
GAGACGAACCAGTTAGTTTGTCATTTCCGGTTTGACGATCATCAGACGATCACCCGTT
TGACTCAGGATTCGCGCGTCGATGACTTACAGCAGATTCACACAGGTATCATGCTTTC
AACGCGTCTGCTCAATGAAGTGGACGATACGGCGCGTAAGAAAAGGGCATGATAATG
AGTGAAAAAAGCATTGTTCAGGAAGCTCGCGATATCCAGTTGGCGATGGAGTTGATT
AATCTTGGCGCTCGTCTACAAATGCTGGAAAGCGAAACACAGCTCAGCCGTGGTCGC
CTCATCAGGCTGTACAAAGAATTACGCGGTAGCCCGCCGCCTAAAGGGATGCTGCCAT
TTTCGACAGACTGGTTTATGACCTGGGAGCAAAATATTCATGCCTCCATGTTCTGCAA
CGCCTGGCAATTTTTACTGAAGACCGGCTTATGCAGCGGTGTGGATGCGGTGATTAAA
GCTTATCGGCTTTATCTTGAGCAGTGTCCGCAACCGCCTGAAGGGCCGTTGTTGGCGC
TGACTCGCGCATGGACGCTGGTGCGTTTTGTTGAAAGTGGGTTGCTTGAATTGTCGA
GCTGTAACTGCTGCGGTGGGAACTTTATTACCCATGCGCATCAGCCCGTAGGCAGCTT
TGCGTGTAGTTTATGCCAGCCGCCATCCCGCGCAGTAAAAAGACGTAAACTTTCCCGA
GATGCTGCCGATATTATTCCACAACTGCTGGATGAACAGATCGAACAGGCTGTTTAA
or at least 90%, 95%, 96%, 97%, 98%, 99% or 100% identity thereto.
Protein sequence
flhD
(SEQ ID NO: 4)
MHTSELLKHIYDINLSYLLLAQRLIVQDKASAMFRLGINEEMANTLGALTLPQMVKLAE
TNQLVCHFRFDDHQTITRLTQDSRVDDLQQIHTGIMLSTRLLNEVDDTARKKRA
or at least 90%, 95%, 96%, 97%, 98%, 99% or 100% identity thereto.
flhC
(SEQ ID NO: 5)
MSEKSIVQEARDIQLAMELINLGARLQMLESETQLSRGRLIRLYKELRGSPPPKGMLPFST
DWFMTWEQNIHASMFCNAWQFLLKTGLCSGVDAVIKAYRLYLEQCPQPPEGPLLALTR
AWTLVRFVESGLLELSSCNCCGGNFITHAHQPVGSFACSLCQPPSRAVKRRKLSRDAADII
PQLLDEQIEQAV
or at least 90%, 95%, 96%, 97%, 98%, 99% or 100% identity
thereto.
Other sequences can also be used to control flagella activity, these include, for example,
motA, motB, flhE, cheZ, cheY cheB, cheR, cheM, cheW, cheA,
fliA, fliY, fliZ, fliB, fliS, fliE, fliF, fliJ, fliL, fliM,
fliN, fliO, flip, fliQ, fliR, fliG, fliH, fliI, fliT, fliD,
fliC, fljB, ycrG, flgN, flgM, flgA, flgB, flgC, flgD, flgE,
flgF, flgG, flgH, flgI, flgJ, flgK and/or flgL.
motA, WP_000906312.1
>WP_000906312.1 MULTISPECIES: flagellar motor stator protein MotA [Salmonella]
(SEQ ID NO: 6)
MLILLGYLVVIGTVFGGYVMTGGHLGALYQPAELVIIGGAGIGAFIVGNNGKAIKGTMK
AIPLLFRRSKYTKSMYMDLLALLYRLMAKSRQQGMFSLERDIENPKESEIFASYPRILAD
AVMLDFIVDYLRLIISGNMNTFEIEALMDEEIETHESEAEVPANSLAMVGDSLPAFGIVAA
VMGVVHALASADRPAAELGALIAHAMVGTFLGILLAYGFISPLATVLRQKSAETTKMMQ
CVKITLLSNLNGYAPPIAVEFGRKTLYSSERPSFIELEEHVRAVRNPNQQQTTEEA
or at least 90%, 95%, 96%, 97%, 98%, 99% or 100% identity thereto.
motB, WP_000795653.1
>WP_000795653.1 MULTISPECIES: flagellar motor protein MotB [Salmonella]
(SEQ ID NO: 7)
MKNQAHPIVVVKRRRHKPHGGGAHGSWKIAYADFMTAMMAFFLVMWLISISSPKELIQI
AEYFRTPLATAVTGGNRIANSESPIPGGGDDYTQQQGEVEKQPNIDELKKRMEQSRLNKL
RGDLDQLIESDPKLRALRPHLKIDLVQEGLRIQIIDSQNRPMFKTGSAEVEPYMRDILRAI
APVLNGIPNRISLAGHTDDFPYANGEKGYSNWELSADRANASRRELVAGGLDNGKVLRV
VGMAATMRLSDRGPDDAINRRISLLVLNKQAEQAILHENAESQNEBVSVLQQPAAAPPA
SVPTSPKAEPR
or at least 90%, 95%, 96%, 97%, 98%, 99% or 100% identity
thereto.
flhE, WP_001233619.1
(SEQ ID NO: 8)
>WP_001233619.1 MULTISPECIES: flagellar protein FlhE [Salmonella]
MRKWLALLLFPLTVQAAGEGAWQDSGMGVTLNYRGVSASSSPLSARQPVSGVMTLVA
WRYELNGPTPAGLRVRLCSQSRCVELDGQSGTTHGFAHVPAVEPLRFVWEVPGGGRLIP
ALKVRSNQVIVNYR
or at least 90%, 95%, 96%, 97%, 98%, 99% or 100%
identity thereto.
cheZ, WP_000983586.1
>WP_000983586.1 MULTISPECIES: protein phosphatase CheZ [Salmonella]
(SEQ ID NO: 9)
MMQPSIKPADEGSAGDIIARIGSLTRMLRDSLRELGLDQAIAEAAEAIPDARDRLDYVVQ
MTAQAAERALNSVEASQPHQDAMEKEAKALTQRWDEWFDNPIELSDARELVTDTRQFL
RDVPGHTSFTNAQLLDIMMAQDFQDLTGQVIKRMMDVIQEIERQLLMVLLENIPEQSAR
PKRENESLLNGPQVDTSKAGVVASQDQVDDLLDSLGF
or at least 90%,
95%, 96%, 97%, 98%, 99% or 100% identity thereto.
cheY WP 000763861.1
>WP_000763861.1 MULTISPECIES: chemotaxis response regulator CheY [Salmonella]
(SEQ ID NO: 10)
MADKELKFLVVDDFSTMRRIVRNLLKELGFNNVEEAEDGVDALNKLQAGGFGFIISDW
NMPNMDGLELLKTIRADSAMSALPVLMVTAEAKKENIIAAAQAGASGYVVKPFTAATL
EEKLNKIFEKLGM
or at least 90%, 95%, 96%, 97%, 98%, 99% or 100%
identity thereto.
cheB, WP_000036392.1
>WP_000036392.1 MULTISPECIES:
protein-glutamate methylesterase/protein glutamine
deamidase [Salmonella]
(SEQ ID NO: 11)
MSKIRVLSVDDSALMRQIMTEIINSHSDMEMVATAPDPLVARDLIKKENPDVLTLDVEMP
RMDGLDFLEKLMRLRPMPVVMVSSLTGKGSEVTLRALELGAIDFVTKPQLGIREGMLAY
SEMIAEK VRTAARARIAAHKPMAAPTTLKAGPLLSSEKLIAIGASTGGTEAIRHVLQPLPL
SSPAVIITQHMPPGFTRSFAERLNKLCQISVKEAEDGERVLPGHAYIAPGDKHMELARSGA
NYQIKIHDGPPVNRHRPSVDVLFHSVAKHAGRNAVGVILTGMGNDGAAGMLAMYQAG
AWTIAQNEASCVVFGMPREAINMGGVSEVVDLSQVSQQMLAKISAGQAIRI
or at least 90%, 95%, 96%, 97%, 98%, 99% or 100% identity thereto.
cheR, WP_000204362.1
>WP_000204362.1 MULTISPECIES: protein-glutamate O-methyltransferase CheR [Salmonella]
(SEQ ID NO: 12)
MTSSLPSGQTSVLLQMTQRLALSDAHFRRICQLIYQRAGIVLADHKRDMVYNRLVRRLR
ALGLDDFGRYLSMLEANQNSAEWQAFINALTTNLTAFFREAHHFPILAEHARRRHGEYR
VWSAAASTGEEPYSIAITLADALGMAPGRWKVFASDIDTEVLEKARSGIYRLSELKTLSP
QQLQRYFMRGTGPHEGLVRVRQELANYVEFSSVNLLEKQYNVPGPFDAIFCRNVMIYFD
KTTQEDILRRFVPLLKPDGLLFAGHSENFSNLVREFSLRGQTVYALSKDKA
or at least 90%, 95%, 96%, 97%, 98%, 99% or 100% identity thereto.
cheM, WP_000483274.1
>WP_000483274.1 MULTISPECIES: methyl-accepting chemotaxis protein II [Salmonella]
(SEQ ID NO: 13)
MFNRIRVVTMLMMVLGVFALLQLVSGGLLFSSLQHNQQGFVISNELRQQQSELTSTWDL
MLQTRINLSRSAARMMMDASNQQSSAKTDLLQNAKTTLAQAAAHYANFKNMTPLPAM
AEASANVDEKYQRYQAALAELIQFLDNGNMDAYFAQPTQGMQNALGEALGNYARVSE
NLYRQTFDQSAHDYRFAQWQLGVLAVVLVLILMVVWFGIRHALLNPLARVITHIREIASG
DLTKTLTVSGRNEIGELAGTVEHMQRSLIDTVTQVREGSDAIYSGTSEIAAGNTDLSSRTE
QQASALEETAASMEQLTATVKQNADNARQASQLAQSASETARHGGKVVDGVVNTMHEI
ADSSKKIADIISVIDGIAFQTNILALNAAVEAARAGEQGRGFAVVAGEVRNLASRSAQAA
KEIKALIEDSVSRVDTGSVLVESAGETMTDIVNAVTRVTDIMGEIASASDEQSRGIDQVAL
AVSEMDRVTQQNASLVQESAAAAAALEEQASRLTQAVSAFRLASRPLAVNKPEMRLSVN
AQSGNTPQSLAARDDANWETF
or at least 90%, 95%, 96%, 97%, 98%, 99%
or 100% identity thereto.
cheW, WP_000147295.1
>WP_000147295.1 MULTISPECIES: chemotaxis protein CheW [Salmonella]
(SEQ ID NO: 14)
MTGMSNVSKLAGEPSGQEFLVFTLGNEEYGIDILKVQEIRGYDQVTRIANTPAFIKGVTN
LRGVIVPIVDLRVKFCEGDVEYDDNTVVIVLNLGQRVVGIVVDGVSDVLSLTAEQIRPAP
EFAVTLSTEYLTGLGALGERMLILVNIEKLLNSEEMALLDIAASHVA
or at
least 90%, 95%, 96%, 97%, 98%, 99% or 100% identity thereto.
cheA, WP_000061302.1
>WP_000061302.1 MULTISPECIES: chemotaxis protein CheA [Salmonella]
(SEQ ID NO: 15)
MSMDISDFYQTFFDEADELLADMEQHLLDLVPESPDAEQLNAIFRAAHSIKGGAGTFGFT
ILQETTHLMENLLDEARRGEMQLNTDIINLFLETKDIMQEQLDAYKNSEEPDAASFEYIC
NALRQLALEAKGETTPAVVETAALSAAIQEESVAETESPRDESKLRIVLSRLKANEVDLLE
EELGNLATLTDVVKGADSLSATLDGSVAEDDIVAVLCFVIEADQIAFEKVVAAPVEKAQE
KTEVAPVAPPAVVAPAAKSAAHEHHAGREKPARERESTSIRVAVEKVDQLINLVGELVITQ
SMLAQRSNELDPVNHGDLITSMGQLQRNARDLQESVMSIRMMPMEYVFSRFPRLVRDL
AGKLGKQVELTLVGSSTELDKSLIERIIDPLTHLVRNSLDHGIEMPEKRLEAGKNVVGNLI
LSAEHQGGNICIEVTDDGAGLNRERILAKAMSQGMAVNENMTDDEVGMLIFAPGFSTAE
QVTDVSGRGVGMDVVKRNIQEMGGHVEIQSKQGSGTTIRILLPLTLAILDGMSVRVAGE
VFILPLNAVMESLQPREEDLHPLAGGERVLEVRGEYLPLVELWKVFDVDGAKTEATQGI
VVILQSAGRRYALLVDQLIGQHQVVVKNLESNYRKVPGISAATILGDGSVALIVDVSALQ
GLNREQRMAITAA
or at least 90%, 95%, 96%, 97%, 98%, 99% or 100%
identity thereto.
fliA, WP_001087453.1
>WP_001087453.1 MULTISPECIES: RNA polymerase sigma factor FliA [Salmonella]
(SEQ ID NO: 16)
MNSLYTAEGVMDKHSLWQRYVPLVRHEALRLQVRLPASVELDDLLQAGGIGLLNAVDR
YDALQGTAFTTYAVQRIRGAMLDELRSRDWVPRSVRRNAREVAQAMGQLEQELGRNAT
ETEVAERLGIPVAEYRQMLLDTNNSQLFSYDEWREEHGDSIELVTEEHQQENPLHQLLEG
DLRQRVMDAIESLPEREQLVLTLYYQEELNLKEIGAVLEVGESRVSQLHSQAIKRLRTKLG
KL
or at least 90%, 95%, 96%, 97%, 98%, 99% or 100% identity thereto.
fliY, WP_000761635.1
>WP_000761635.1 MULTISPECIES: cystine ABC transporter substrate-binding protein
[Salmonella]
(SEQ ID NO: 17)
MKLALLGRQALMGVMAVALVAGMSAKSFADEGLLNKVKERGTLLVGLEGTYPPFSFQG
EDGKLTGFEVDFAEALAKHLGVKASLKPTKWDGMLASLDAKRIDVVINQVTISDVRKK
KYDFSTPYTVSGIQALVKKGNEGTIKTAADLQGKKVGVGLGTNYEEWLRQHVQGVDIR
TYDDDPTKYQDLRVGRIDAILVDRLAALDLVKKTKGTLAVTGDAFSRQESGVALRKGNE
DLLKAVDNAIAEMQKDGTLKALSEKWFGADVTQ
or at least 90%, 95%,
96%, 97%, 98%, 99% or 100% identity thereto.
fliZ, WP_000218080.1
>WP_000218080.1 MULTISPECIES: flagella biosynthesis regulatory protein FliZ [Salmonella]
(SEQ ID NO: 18)
MTVQQPKRRPLSRYLKDFKHSQTHCAHCHKLLDRITLVRRGKIVNKIAISQLDMLLDDA
AWQREQKEWVALCRFCGDLHCKKQSDFFDIIGFKQYLFEQTEMSHGTVREYVVRLRRL
GNYLSEQNISHDLLQDGFLDESLAPWLPETSTNNYRIALRKYQQYKAHQQIAPRQKSPFT
ASSDIY
or at least 90%, 95%, 96%, 97%, 98%, 99% or 100% identity thereto.
fliB, WP_000079794.1
>WP_000079794.1 MULTISPECIES: FliC/FljB family flagellin [Salmonella]
(SEQ ID NO: 19)
MAQVINTNSLSLLTQNNLNKSQSALGTAIERLSSGLRINSAKDDAAGQAIANRFTANIKG
LTQASRNANDGISIAQTTEGALNEINNNLQRVRELAVQSANSTNSQSDLDSIQAEITQRLN
EIDRVSGQTQFNGVKVLAQDNTLTIQVGANDGETIDIDLKQINSQTLGLDSLNVQKAYDV
KDTAVTTKAYANNGTTLDVSGLDDAAIKAATGGINGTASVTGGAVKFDADNNKYFVTI
GGFTGADAAKNGDYEVNVATDGTVTLAAGATKTTMPAGATTKTEVQELKDTPAVVSAD
AKNALIAGGVDATDANGAELVKMSYTDKNGKTIEGGYALKAGDKYYAADYDEATGAI
KAKTTSYTAADGTTKTAANQLGGVDGKTEVVTIDGKTYNASKAAGHDFKAQPELAEA
AAKTTENPLQKIDAALAQVDALRSDLGAVQNRFNSAITNLGNTVNNLSEARSRIEDSDY
ATEVSNMSRAQILQQAGTSVLAQANQVPQNVLSLLR
or at least 90%,
95%, 96%, 97%, 98%, 99% or 100% identity thereto.
fliS, WP_000287764.1
>WP_000287764.1 MULTISPECIES: flagellar export chaperone FliS [Salmonella]
(SEQ ID NO: 20)
MYTASGIKAYAQVSVESAVMSASPHQLIEMLFDGANSALVRARLFLEQGDVVAKGEALS
KAINIIDNGLKAGLDQEKGGEIATNLSELYDYMIRRLLQANLRNDAQAIEEVEGLLSNIAE
AWKQISPKASFQESR
or at least 90%, 95%, 96%, 97%, 98%, 99% or 100%
identity thereto.
fliE, WP_000719036.1
>WP_000719036.1 MULTISPECIES: flagellar hook-basal body complex protein FliE
[Salmonella]
(SEQ ID NO: 21)
MAAIQGIEGVISQLQATAMAARGQDTHSQSTVSFAGQLHAALDRISDRQAAARVQAEKF
TLGEPGIALNDVMADMQKASVSMQMGIQVRNKLVAAYQEVMSMQV
or at least 90%, 95%, 96%, 97%, 98%, 99% or 100% identity thereto.
fliF, WP_001276834.1
>WP_001276834.1 MULTISPECIES: flagellar M-ring protein FliF [Salmonella]
(SEQ ID NO: 22)
MSATASTATQPKPLEWLNRLRANPRIPLIVAGSAAVAIVVAMVLWAKTPDYRTLFSNLSD
QDGGAIVAQLTQMNIPYRFANGSGAIEVPADKVHELRLRLAQQGLPKGGAVGFELLDQE
KFGISQFSEQVNYQRALEGELARTIETLGPVKSARVHLAMPKPSLFVREQKSPSASVTVT
LEPGRALDEGQISAVVHLVSSAVAGLPPGNVTLVDQSGHLLTQSNTSGRDLNDAQLKFAN
DVESRIQRRIEAILSPIVGNGNVHAQVTAQLDFANKEQTEEHYSPNGDASKATLRSRQLNI
SEQVGAGYPGGVPGALSNQPAPPNEAPIATPPTNQQNAQNTPQTSTSTNSNSAGPRSTQR
NETSNYEVDRTIRHTKMNVGDIERLSVAVVVNYKTLADGKPLPLTADQMKQIEDLTREA
MGFSDKRGDTLNVVNSPFSAVDNTGGELPFWQQQSFIDQLLAAGRWLLVLVVAWILWR
KAVRPQLTRRVEEAKAAQEQAQVRQETEEAVEVRLSKDEQLQQRRANQRLGAEVMSQR
IREMSDNDPRVVALVIRQWMSNDHE
or at least 90%, 95%, 96%, 97%, 98%,
99% or 100% identity thereto.
fliJ, WP_000046981.1
>WP_000046981.1 MULTISPECIES: flagella biosynthesis chaperone FliJ [Salmonella]
(SEQ ID NO: 23)
MAQHGALETLKDLAEKEVDDAARLLGEMRRGCQQAEEQLKMLIDYQNEYRSNLNTD
MGNGIASNRWINYQQFIQTLEKAIEQHRLQLTQWTQKVDLALKSWREKKQRLQAWQTL
QDRQTAAALLAENRMDQKKMDEFAQRAAMRKPE
or at least 90%, 95%,
96%, 97%, 98%, 99% or 100% identity thereto.
fliL, WP_000132169.1
>WP_000132169.1 MULTISPECIES: flagellar basal body-associated protein FliL [Salmonella]
(SEQ ID NO: 24)
MTDSAINKKSKRSIWIPLLVLITLAACATAGYSYWRMQQQPTTNAKAEPAPPPAPVFFAL
DTFTVNLGDADRVLYIGVTLRLKDEATRARLNEYLPEVRSRLLLLFSRQNAAELSTEAGK
QKLIAAIKETLAAPLVAGQPKQVVTDVLYTAFILR
or at least 90%, 95%,
96%, 97%, 98%, 99% or 100% identity thereto.
fliM, WP_000502811.1
>WP_000502811.1 MULTISPECIES: flagellar motor switch protein FliM [Salmonella]
(SEQ ID NO: 25)
MGDSILSQAEIDALLNGDSDTKDEPTPGIASDSDIRPYDPNTQRRVVRERLQALEIINERF
ARQFRMGLFNLLRRSPDITVGAIRIQPYHEFARNLPVPTNLNLIHLKPLRGTGLVVFSPSL
VFIAVDNLFGGDGRFPTKVEGREFTHTEQRVINRMLKLALEGYSDAWKAINPLEVEYVR
SEMQVKFTNITTSPNDIVVNTPFHVEIGNLTGEFNICLPFSMIEPLRELLVNPPLENSRHED
QNWRDNLVRQVQHSELELVANFADIPLRLSQILKLKPGDVLPIEKPDRIIAHVDGVPVLTS
QYGTVNGQYALRVEHLINPILNSLNEEQPK
or at least 90%, 95%, 96%,
97%, 98%, 99% or 100% identity thereto.
fliN, WP_001282115.1
>WP_001282115.1 MULTISPECIES: flagellar motor switch protein FliN [Salmonella]
(SEQ ID NO: 26)
MSDMNNPSDENTGALDDLWADALNEQKATTTKSAADAVFQQLGGGDVSGAMQDIDLI
MDIPVKLTVELGRTRMTIKELLRLTQGSVVALDGLAGEPLDILINGYLIAQGEVVVVADK
YGVRITDIITPSERMRRLSR
or at least 90%, 95%, 96%, 97%, 98%, 99% or
100% identity thereto.
fliO, WP_000978276.1
>WP_000978276.1 MULTISPECIES: flagellar type III secretion system protein FliO
[Salmonella]
(SEQ ID NO: 27)
MMKTEATVSQPTAPAGSPLMQVSGALIGIIALILAAAWVIKRMGFAPKGNSVRGLKVSAS
ASLGPRERVVIVEVENARLVLGVTASQINLLHTLPPAENDTEAPVAPPADFQNMMKSLLK
RSGRS
or at least 90%, 95%, 96%, 97%, 98%, 99% or 100% identity thereto.
fliP, WP_001253410.1
>WP_001253410.1 MULTISPECIES: flagellar type III secretion system pore protein FliP
[Salmonella]
(SEQ ID NO: 28)
MRRLLFLSLAGLWLFSPAAAAQLPGLISQPLAGGGQSWSLSVQTLVFITSLTFLPAILLMM
TSFTRIIIVFGLLRNALGTPSAPPNQVLLGLALFLTFFIMSPVIDKIYVDAYQPFSEQKISMQ
EALDKGAQPLRAFMLRQTREADLALFARLANSGPLQGPEAVPMRILLPAYVTSELKTAFQ
IGFTIFIPFLIIDLVIASVLMALGMMMVPPATIALPFKLMLFVLVDGWQLLMGSLAQSFYS
or at least 90%, 95%, 96%, 97%, 98%, 99% or 100% identity thereto.
fliQ, WP_000187355.1
>WP_000187355.1 MULTISPECIES: flagellar biosynthesis protein FliQ [Salmonella]
(SEQ ID NO: 29)
MTPESVMMMGTEAMKVALALAAPLLLVALITGLIISILQAATQINEMTLSFIPKIVAVFIAII
VAGPWMLNLLLDYVRTLFSNLPYIIG
or at least 90%, 95%, 96%, 97%,
98%, 99% or 100% identity thereto.
fliR, WP_000616953.1
>WP_000616953.1 MULTISPECIES: flagellar type III secretion system protein
FliR [Salmonella]
(SEQ ID NO: 30)
MIQVTSEQWLYWLHLYFWPLLRVLALISTAPILSERAIPKRVKLGLGIMITLVIAPSLPAND
TPLFSIAALWLAMQQILIGIALGFTMQFAFAAVRTAGEFIGLQMGLSFATFVDPGSHLNMP
VLARIMDMLAMLLFLTFNGHLWLISLLVDTFHTLPIGSNPVNSNAFMALARAGGLIFLNG
LMLALPVITLLLTLNLALGLLNRMAPQLSIFVIGFPLTLTVGIMLMAALMPLIAPFCEHLFS
EIFNLLADIVSEMPINNNP
or at least 90%, 95%, 96%, 97%, 98%, 99% or
100% identity thereto.
fliG, WP_000067735.1
>WP_000067735.1 MULTISPECIES: flagellar motor switch protein FliG [Salmonella]
(SEQ ID NO: 31)
MSNLSGTDKSVILLMTIGEDRAAEVFKHLSTREVQALSTAMANVRQISNKQLTDVLSEFE
QEAEQFAALNINANEYLRSVLVKALGEERASSLLEDILETRDTTSGIETLNFMEPQSAADL
IRDEHPQIIATILVHLKRSQAADILALFDERLRHDVMLRIATFGGVQPAALAELTEVLNGLL
DGQNLKRSKMGGVRTAAEIINLMKTQQEEAVITAVREFDGELAQKIIDEMFLFENLVDVD
DRSIQRLLQEVDSESLLIALKGAEPPLREKFLRNMSQRAADILRDDLANRGPVRLSQVEN
EQKAILLIVRRLAETGEMVIGSGEDTYV
or at least 90%, 95%, 96%, 97%,
98%, 99% or 100% identity thereto.
fliH, WP_000064163.1
>WP_000064163.1 MULTISPECIES: flagellar assembly protein FliH [Salmonella]
(SEQ ID NO: 32)
MSNELPWQVWTPDDLAPPPETFVPVEADNVTLTEDTPEPELTAEQQLEQELAQLKIQAH
EQGYNAGLAEGRQKGHAQGYQEGLAQGLEQGQAQAQTQQAPIHARMQQLVSEFQNTL
DALDSVIASRLMQMALEAARQVIGQTPAVDNSALIKQIQQLLQQEPLFSGKPQLRVHPDD
LQRVEEMLGATLSLHGWRLRGDPTLHHGGCKVSADEGDLDASVATRWQELCRLAAPG
VL
or at least 90%, 95%, 96%, 97%, 98%, 99% or 100% identity thereto.
fliI, WP_ 000213257.1
>WP_000213257.1 MULTISPECIES: flagellum-specific ATP synthase FliI [Salmonella]
(SEQ ID NO: 33)
MTTRLTRWLTALDNFEAKMALLPAVRRYGRLTRATGLVLEATGLQLPLGATCIIERQDGP
ETKEVESEVVGFNGQRLFLMPLEEVEGILPGARVYARNGHGDGLQSGKQLPLGPALLGR
VLDGGGKPLDGLPAPDTLETGALITPPFNPLQRTPIEHVLDTGVRAINALLTVGRGQRMG
LFAGSGVGKSVLLGMMARYTRADVIVVGLIGERGREVKDFIENILGPDGRARSVVIAAP
ADVSPLLRMQGAAYATRIAEDFRDRGQHVLLIMDSLTRYAMAQREIALAIGEPPATKGYP
PSVFAKLPALVERAGNGIHGGGSITAFYTVLTEGDDQQDPIADSARAILDGHIVLSRRLAE
AGHYPAIDIEASISRAMTALITEQHYARVRLFKQLLSSFQRNRDLVSVGAYAKGSDPMLD
KAITLWPQLEAFLQQGIFERADWEDSLQALDLIFPTV
or at least 90%,
95%, 96%, 97%, 98%, 99% or 100% identity thereto.
fliT, WP_000204899.1
>WP_000204899.1 MULTISPECIES: flagella biosynthesis regulatory protein FliT [Salmonella]
(SEQ ID NO: 34)
MTSTVEFINRWQRIALLSQSLLELAQRGEWDLLLQQEVSYLQSIETVMEKQTPPGITRSIQ
DMVAGYIKQTLDNEQLLKGLLQQRLDELSSLIGQSTRQKSLNNAYGRLSGMLLVPDAPG
AS
or at least 90%, 95%, 96%, 97%, 98%, 99% or 100% identity thereto.
fliD, WP_000146802.1
>WP_000146802.1 MULTISPECIES: flagellar filament capping protein FliD [Salmonella]
(SEQ ID NO: 35)
MASISSLGVGSNLPLDQLLTDLTKNEKGRLTPITKQQSANSAKLTAYGTLKSALEKFQTAN
TALNKADLFKSTVASSTTEDLKVSTTAGAAAGTYKINVTQLAAAQSLATKTTFATTKEQL
GDTSVTSRTIKIEQPGRKEPLEIKLDKGDTSMEAIRDAINDADSGIAASIVKVKENEFQLV
LTANSGTDNTMKITVEGDTKLNDLLAYDSTTNTGNMQELVKAENAKLNVNGIDIERQSN
TVTDAPQGITLTLTKKVTDATVTVTKDDTKAKEAIKSWVDAYNSLVDTFSSLTKYTAVEP
GEEASDKNGALLGDSVVRTIQTGIRAQFANSGSNSAFKTMAEIGITQDGTSGKLKIDDDK
LTKVLKDNTAAARELLVGDGKETGITTKIATEVKSYLADDGIIDNAQDNVNATLKSLTKQ
YLSVSNSIDETVARYKAQFTQLDTMMSKLNNTSSYLTQQFTAMNKS
or
at least 90%, 95%, 96%, 97%, 98%, 99% or 100% identity thereto.
fliC, WP 000079805.1
>WP_000079805.1 MULTISPECIES: FliC/FljB family flagellin [Salmonella]
(SEQ ID NO: 36)
MAQVINTNSLSLLTQNNLNKSQSALGTAIERLSSGLRINSAKDDAAGQAIANRFTANIKG
LTQASRNANDGISIAQTTEGALNEINNNLQRVRELAVQSANSTNSQSDLDSIQAEITQRLN
EIDRVSGQTQFNGVKVLAQDNTLTIQVGANDGETIDIDLKQINSQTLGLDTLNVQQKYK
VSDTAATVTGYADTTIALDNSTFKASATGLGGTDQKIDGDLKFDDTTGKYYAKVTVTGG
TGKDGYYEVSVDKTNGEVTLAGGATSPLTGGLPATATEDVKNVQVANADLTEAKAALTA
AGVTGTASVVKMSYTDNNGKTIDGGLAVKVGDDYYSATQNKDGSISINTTKYTADDGT
SKTALNKLGGADGKTEVVSIGGKTYAASKAEGHNFKAQPDLAEAAATTTENPLQKIDAA
LAQVDTLRSDLGAVQNRFNSAITNLGNTVNNLTSARSRIEDSDYATEVSNMSRAQILQQA
GTSVLAQANQVPQNVLSLLR
or at least 90%, 95%, 96%, 97%, 98%, 99%
or 100% identity thereto.
fljB, WP_000079794.1
>WP_000079794.1 MULTISPECIES: FliC/FljB family flagellin [Salmonella]
(SEQ ID NO: 37)
MAQVINTNSLSLLTQNNLNKSQSALGTAIERLSSGLRINSAKDDAAGQAIANRFTANIKG
LTQASRNANDGISIAQTTEGALNEINNNLQRVRELAVQSANSTNSQSDLDSIQAEITQRLN
EIDRVSGQTQFNGVKVLAQDNTLTIQVGANDGETIDIDLKQINSQTLGLDSLNVQKAYDV
KDTAVTTKAYANNGTTLDVSGLDDAAIKAATGGTNGTASVTGGAVKFDADNNKYFVTI
GGFTGADAAKNGDYEVNVATDGTVTLAAGATKTTMPAGATTKTEVQELKDTPAVVSAD
AKNALIAGGVDATDANGAELVKMSYTDKNGKTIEGGYALKAGDKYYAADYDEATGAI
KAKTTSYTAADGTTKTAANQLGGVDGKTEVVTIDGKTYNASKAAGHDFKAQPELAEA
AAKTTENPLQKIDAALAQVDALRSDLGAVQNRFNSAITNLGNTVNNLSEARSRIEDSDY
ATEVSNMSRAQILQQAGTSVLAQANQVPQNVLSLLR
or at least 90%,
95%, 96%, 97%, 98%, 99% or 100% identity thereto.
flgN, WP_000197547.1
>WP_000197547.1 MULTISPECIES: flagella biosynthesis chaperone FlgN [Salmonella]
(SEQ ID NO: 38)
MTRLSEILDQMTTVLNDLKTVMDAEQQQLSVGQINGSQLQRITEEKSSLLATLDYLEQQ
RRLEQNAQRSANDDIAERWQAITEKTQHLRDLNQHNGWLLEGQIERNQQALEVLKPHQ
EPTLYGADGQTSVSHRGGKKISI
or at least 90%, 95%, 96%, 97%, 98%,
99% or 100% identity thereto.
flgM, WP_000020893.1
>WP_000020893.1 MULTISPECIES: anti-sigma-28 factor FlgM [Salmonella]
(SEQ ID NO: 39)
MSIDRTSPLKPVSTVQTRETSDTPVQKTRQEKTSAATSASVTLSDAQAKLMQPGVSDINM
ERVEALKTAIRNGELKMDTGKIADSLIREAQSYLQSK
or at least 90%,
95%, 96%, 97%, 98%, 99% or 100% identity thereto.
flgA, WP_001194082.1
>WP_001194082.1 MULTISPECIES: flagellar basal body P-ring formation protein FlgA
[Salmonella]
(SEQ ID NO: 40)
MQTLKRGFAVAALLFSPLTMAQDINAQLTTWFSQRLAGFSDEVVVTLRSSPNLLPSCEQP
AFSMTGSAKLWGNVNVVARCANEKRYLQVNVQATGNYVAVAAPIARGGKLTPANVTLK
RGRLDQLPPRTVLDIRQIQDAVSLRDLAPGQPVQLTMIRQAWRVKAGQRVQVIANGEGF
SVNAEGQAMNNAAVAQNARVRMTSGQIVSGTVDSDGNILINL
or at least
90%, 95%, 96%, 97%, 98%, 99% or 100% identity thereto.
flgB, WP_000887043.1
>WP_000887043.1 MULTISPECIES: flagellar basal body rod protein FlgB [Salmonella]
(SEQ ID NO: 41)
MLDRLDAALRFQQEALNLRAQRQEILAANIANADTPGYQARDIDFASELKKVMVRGRE
ETGGVALTLTSSHHIPAQAVSSPAVDLLYRVPDQPSLDGNTVDMDRERTQFADNSLKYQM
GLTVLGSQLKGMMNVLQGGN
or at least 90%, 95%, 96%, 97%, 98%, 99%
or 100% identity thereto.
flgC, WP_001196448.1
>WP_001196448.1 MULTISPECIES: flagellar basal body rod protein FlgC [Salmonella]
(SEQ ID NO: 42)
MALLNIFDIAGSALAAQSKRLNVAASNLANADSVTGPDGQPYRAKQVVFQVDAAPGQA
TGGVKVASVIESQAPEKLVYEPGNPLADANGYVKMPNVDVVGEMVNTMSASRSYQANI
EVLNTVKSMMLKTLTLGQ
or at least 90%, 95%, 96%, 97%, 98%, 99% or
100% identity thereto.
flgD, WP_000020450.1
>WP_000020450.1 MULTISPECIES: flagellar hook assembly protein FlgD [Salmonella]
(SEQ ID NO: 43)
MSIAVNMNDPTNTGVKTTTGSGSMTGSNAADLQSSFLTLLVAQLKNQDPTNPLQNNELT
TQLAQISTVSGIEKLNTTLGAISGQIDNSQSLQATTLIGHGVMVPGTTILAGKGAEEGAVT
STTPFGVELQQPADKVTATITDKDGRVVRTLEIGELRAGVHTFTWDGKQTDGTTVPNGS
YNIAITASNGGTQLVAQPLQFALVQGVTKGSNGNLLDLGTYGTTTLDEVRQII
or at least 90%, 95%, 96%, 97%, 98%, 99% or 100% identity thereto.
flgE, WP_000010567.1
>WP_000010567.1 MULTISPECIES: flagellar hook protein FlgE [Salmonella]
(SEQ ID NO: 44)
MSFSQAVSGLNAAATNLDVIGNNIANSATYGFKSGTASFADMFAGSKVGLGVKVAGITQ
DFTDGTTTNTGRGLDVAISQNGFFRLVDSNGSVFYSRNGQFKLDENRNLVNMQGMQLT
GYPATGTPPTIQQGANPAPITIPNTLMAAKSTTTASMQINLNSTDPVPSKTPFSVSDADSY
NKKGTVTVYDSQGNAHDMNVYFVKTKDNEWAVYTHDSSDPAATAPTTASTTLKFNEN
GILESGGTVNITTGTINGATAATFSLSFLNSMQQNTGANNIVATNQNGYKPGDLVSYQINN
DGTVVGNYSNEQEQVLGQIVLANFANNEGLASQGDNVWAATQASGVALLGTAGSGNFG
KLINGALEASNVDLSKELVNMIVAQRNYQSNAQTIKTQDQILNTLVNLR
or at least 90%, 95%, 96%, 97%, 98%, 99% or 100% identity thereto.
flgF, WP_000349278.1
>WP_000349278.1 MULTISPECIES: flagellar basal body rod protein FlgF [Salmonella]
(SEQ ID NO: 45)
MDHAIYTAMGAASQTLNQQAVTASNLANASTPGFRAQLNALRAVPVDGLSLATRTLVTA
STPGADMTPGQLDYTSRPLDVALQQDGWLVVQAADGAEGYTRNGNIQVGPTGQLTIQG
HPVIGEGGPITVPEGSEITIAADGTISALNPGDPPNTVAPVGRLKLVKAEGNEVQRSDDGL
FRLTAEAQAERGAVLAADPSIRIMSGVLEGSNVKPVEAMTDMIANARRFEMQMKVITSV
DENEGRANQLLSMS
or at least 90%, 95%, 96%, 97%, 98%, 99% or 100%
identity thereto.
flgG, WP_000625851.1
>WP_000625851.1 MULTISPECIES: flagellar basal-body rod protein FlgG [Salmonella]
(SEQ ID NO: 46)
MISSLWIAKTGLDAQQTNMDVIANNLANVSTNGFKRQRAVFEDLLYQTIRQPGAQSSEQ
TTLPSGLQIGTGVRPVATERLHSQGNLSQTNNSKDVAIKGQGFFQVMLPDGTSAYTRDGS
FQVDQNGQLVTAGGFQVQPAITIPANALSITIGRDGVVSVTQQGQAAPVQVGQLNLTTFM
NDTGLESIGENLYIETQSSGAPNESTPGLNGAGLLYQGYVETSNVNVAEELVNMIQVQRA
YEINSKAVSTTDQMLQKLTQL
or at least 90%, 95%, 96%, 97%, 98%, 99%
or 100% identity thereto.
flgH, WP_001174897.1
>WP_001174897.1 MULTISPECIES: flagellar basal body L-ring protein FlgH [Salmonella]
(SEQ ID NO: 47)
MQKYALHAYPVMALMVATLTGCAWIPAKPLVQGATTAQPIPGPVPVANGSIFQSAQPINY
GYQPLFEDRRPRNIGDTLTIVLQENVSASKSSSANASRDGKTSFGFDTVPRYLQGLFGNS
RADMEASGGNSFNGKGGANASNTFSGTLTVTVDQVLANGNLHVVGEKQIAINQGTEFI
RFSGVVNPRTISGSNSVPSTQVADARIEYVGNGYINEAQNMGWLQRFFLNLSPM
or at least 90%, 95%, 96%, 97%, 98%, 99% or 100% identity thereto.
flgI, WP_001518955.1
>WP_001518955.1 MULTISPECIES: flagellar basal body P-ring protein FlgI [Salmonella]
(SEQ ID NO: 48)
MFKALAGIVLALVATLAHAERIRDLTSVQGVRENSLIGYGLVVGLDGTGDQTTQTPFTTQ
TLNNMLSQLGITVPTGTNMQLKNVAAVMVTASYPPFARQGQTIDVVVSSMGNAKSLRG
GTLLMTPLKGVDSQVYALAQGNILVGGAGASAGGSSVQVNQLNGGRITNGAIIERELPT
QFGAGNTINLQLNDEDFTMAQQITDAINRARGYGSATALDARTVQVRVPSGNSSQVRFL
ADIQNMEVNVTPQDAKVVINSRTGSVVMNREVTLDSCAVAQGNLSVTVNRQLNVNQPN
TPFGGGQTVVTPQTQIDLRQSGGSLQSVRSSANLNSVVRALNALGATPMDLMSILQSMQ
SAGCLRAKLEII
or at least 90%, 95%, 96%, 97%, 98%, 99% or 100% identity
thereto.
flgJ, WP_000578692.1
>WP_000578692.1 MULTISPECIES: flagellar assembly peptidoglycan hydrolase FlgJ
[Salmonella]
(SEQ ID NO: 49)
MIGDGKLLASAAWDAQSLNELKAKAGQDPAANIRPVARQVEGMFVQMMLKSMREALP
KDGLFSSDQTRLYTSMYDQQIAQQMTAGKGLGLADMMVKQMTSGQTMPADDAPQVP
LKFSLETVNSYQNQALTQLVRKAIPKTPDSSDAPLSGDSKDFLARLSLPARLASEQSGVP
HHLILAQAALESGWGQRQILRENGEPSYNVFGVKATASWKGPVTEITTTEYENGEAKKV
KAKFRVYSSYLEALSDYVALLTRNPRYAAVTTAATAEQGAVALQNAGYATDPNYARKLTS
MIQQLKAMSEKVSKTYSANLDNLF
or at least 90%, 95%, 96%, 97%, 98%,
99% or 100% identity thereto.
flgK, WP_000096425.1
>WP_000096425.1 MULTISPECIES: flagellar hook-associated protein FlgK [Salmonella]
(SEQ ID NO: 50)
MSSLINHAMSGLNAAQAALNTVSNNINNYNVAGYTRQTTILAQANSTLGAGGWIGNGV
YVSGVQREYDAFITNQLRGAQNQSSGLTTRYEQMSKIDNLLADKSSSLSGSLQSFFTSLQ
TLVSNAEDPAARQALIGKAEGLVNQFKTTDQYLRDQDKQVNIAIGSSVAQINNYAKQIAN
LNDQISRMTGVGAGASPNDLLDQRDQLVSELNKIVGVEVSVQDGGTYNLTMANGYTLV
QGSTARQLAAVPSSADPTRTTVAYVDEAAGNIEIPEKLLNTGSLGGLLTFRSQDLDQTRNT
LGQLALAFADAFNAQHTKGYDADGNKGKDFFSIGSPVVYSNSNNADKTVSLTAKVVDS
TKVQATDYKIVFDGTDWQVTRTADNTTFTATKDADGKLEIDGLKVTVGTGAQKNDSFL
LKPVSNAIVDMNVKVTNEAEIAMASESKLDPDVDTGDSDNRNGQALLDLQNSNVVGG
NKTFNDAYATLVSDVGNKTSTLKTSSTTQANVVKQLYKQQQSVSGVNLDEEYGNLQRY
QQYYLANAQVLQTANALFDALLNIR
or at least 90%, 95%, 96%, 97%,
98%, 99% or 100% identity thereto.
flgL WP_001223033.1
>WP_001223033.1 MULTISPECIES: flagellar hook-associated protein FlgL [Salmonella]
(SEQ ID NO: 51)
MRISTQMMYEQNMSGITNSQAEWMKLGEQMSTGKRVTNPSDDPIAASQAVVLSQAQA
QNSQYALARTFATQKVSLEESVLSQVTTAIQTAQEKIVYAGNGTLSDDDRASLATDLQGI
RDQLMNLANSTDGNGRYIFAGYKTEAAPFDQATGGYHGGEKSVTQQVDSARTMVIGHT
GAQIFNSITSNAVPEPDGSDSEKNLFVMLDTAIAALKTPVEGNNVEKEKAAAAIDKTNRG
LKNSLNNVLTVRAELGTQLSELSTLDSLGSDRALGQKLQMSNLVDVDWNSVISSYVMQ
QAALQASYKTFTDMQGMSLFQLNR
or at least 90%, 95%, 96%, 97%, 98%,
99% or 100% identity thereto.

### II. Vectors/Plasmids

In the present compositions and/or methods, DNA, RNA (e.g., a nucleic acid-based gene interfering agent) or protein may be produced by recombinant methods. The nucleic acid is inserted into a replicable vector for expression. Many such vectors are available. The vector components generally include, but are not limited to, one or more of the following: an origin of replication, one or more marker genes, an enhancer element, a promoter, and a transcription termination sequence and coding sequence. In some embodiments, for example in the utilization of bacterial delivery agents such as Salmonella, the gene and/or promoter (a sequence of interest) may be integrated into the host cell chromosome or may be presented on, for example, a plasmid/vector.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors can contain a promoter that is recognized by the host organism and is operably linked to the nucleic acid sequence, such as a nucleic acid sequence coding for an open reading frame. Promoters are untranslated sequences located upstream (5′) to the start codon of a structural gene (generally within about 100 to 1000 bp) that control the transcription of particular nucleic acid sequence to which they are operably linked. In bacterial cells, the region controlling overall regulation can be referred to as the operator. Promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Promoters suitable for use with prokaryotic hosts include the β-lactamase and lactose promoter systems, alkaline phosphatase, a tryptophan (trp) promoter system, hybrid promoters such as the tac promoter, and starvation promoters (Matin, A. (1994) Recombinant DNA Technology II, Annals of New York Academy of Sciences, 722:277-291). However, other known bacterial promoters are also suitable. Such nucleotide sequences have been published, thereby enabling a skilled worker to operably ligate them to a DNA coding sequence. Promoters for use in bacterial systems also can contain a Shine-Dalgarno (S.D.) sequence operably linked to the coding sequence.

Construction of suitable vectors containing one or more of the above-listed components employs standard ligation techniques. Isolated plasmids or DNA fragments are cleaved, tailored, and re-ligated in the form desired to generate the plasmids required.

In some embodiments of the invention, the expression vector is a plasmid or bacteriophage vector suitable for use in Salmonella, and the DNA, RNA and/or protein is provided to a subject through expression by an engineered Salmonella (in one aspect attenuated) administered to the patient. The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides and may be chemically modified by such means as methylation or the inclusion of protecting groups or cap- or tail structures.

One embodiment provides a Salmonella strain comprising a lysis gene (coding for a lysis protein) or cassette operably linked to an inducible promoter. In one embodiment, the inducible promoter is the salicylic acid inducible promoter (CCAGCTGATCAGTTGTAGAATGAAAAAATAAGTGCCTGCCAGAATCAAAAAAGTTCC AGCCAAAAATAGATATTGCATCAAACCTTGGAATGAGCTCAATTCAATCAGTCGTTCA TAAAAACGAGCATTTGTAAAACTTGCTAACCAACAAGCTAGAAGCCCATTAAATGTAT TTATAGATAAACTTACTTTTTTGAATTGTAGCAATTTAAACATCTCAATATCGTTCAGTT ATTTGAGGGGTATAGCTACATTAAAGAGTATGAAAATTAAGAGAAAATTAGTTTTCGC TTCAATGATTCAATTTAAATCTAAATATCAATGTTTTAAGATCATAATGATGGATATGAA AACTTAAACCTATACCGGATAGGCAATTAAGAAAGAAATATGATATAATTGTTTTAAGA TATTGATTTTAATAAATTATTTAAAAAATTAATAACTGCTTTAAAAAACTTTAAATTGGA TTTAATTGAGTTTGCAGACTAAAAATAAAACAATAAATCAAGTTCTCAAAGGAAATGA GTC (Accession number: CR543861.1), (SEQ ID NO: 52) or at least 90%, 95%, 96%, 97%, 98%, 99% or 100% identity thereto.

In one embodiment, the lysis gene/cassette is Lysin E from phage phiX174 (in one embodiment, the lysin gene E contains a C-terminal ssrA degradation sequence of AANDENYALAA (SEQ ID NO: 53); AANDENYADAS (SEQ ID NO: 54); or AANDENYNYADAS (SEQ ID NO: 55)), phage iEPS5, or lambda phage.

salR regulatory protein (a regulator protein that activates pSal)

NT sequence:
(SEQ ID NO: 56)
ATGGACCTGTCTCTGATCCGTATCTTCATCTGCGTTTACGAAAACAAAAACATCTCTAA
AGCGGCGGAAATCCTGAACCTGTCTCAGCCGTCTGTTACCTACAACCTGAACCGTCT
GCGTAAACACCTGAACAACCCGCTGTTCGAACGTACCCAGTACGGTGTTGAAGCGAC
CAAACTGTCTCACGAACTGTACCCGGTTTTCAAAGAATCTATCCTGAAAATCGAAATC
GCGGTTGACGAAGCGCTGAACTTCAACCCGCTGACCTCTAACAAAACCTTCCGTATC
GGTCTGTCTGACATCGGTGAAATCTGCCTGCTGCCGACCCTGATCGAATACCTGCGTG
CGCACGCGCCGAAAATCAAAATCGAAGTTGAAGAAATCAAAATCGACCAGGTTGAA
AAATGGCTGATCGAAGGTTTCATCGACGTTGCGGTTTTCAACTCTACCCACCTGGAAT
TCAAACACCTGGAATACGAAACCCTGTTCCTGGAACGTTACGTTGCGCTGGTTAACAT
GAACCACCCGCGTATCCGTTCTACCCTGTCTTTCGACGCGTACCTGAACGAATCTCAC
GTTGCGATCAAATCTTCTACCGGTCACACCCAGGTTGACCACGTTCTGAAACTGATGG
GTCACCAGCGTAAAATCGCGCTGGAAGTTCCGCACTTCGGTGTTCTGCAGGGTGTTC
TGGACAAAACCGACCTGATGGTTACCCTGCCGTCTCGTGCGGCGCAGCAGTACCTGA
ACCAGTCTCACGTTCGTGTTCTGGAACTGCCGTTCCAGATGTCTGAATTCTACGTTGG
TCTGCACTGGTTCGCGCAGACCAACGAACCGCTGGCGCGTATCTGGCTGATCCAGAC
CTGCAAAAAAGTTATCTCTGTTCTGTAA
or at least 90%, 95%, 96%, 97%,
98%, 99% or 100% identity thereto.
AA sequence:
(SEQ ID NO: 57)
MDLSLIRIFICVYENKNISKAAEILNLSQPSVTYNLNRLRKHLNNPLFERTQYGVEATKLS
HELYPVFKESILKIEIAVDEALNFNPLTSNKTFRIGLSDIGEICLLPTLIEYLRAHAPKIKIEV
EEIKIDQVEKWLIEGFIDVAVFNSTHLEFKHLEYETLFLERYVALVNMNHPRIRSTLSFDAY
LNESHVAIKSSTGHTQVDHVLKLMGHQRKIALEVPHFGVLQGVLDKTDLMVTLPSRAA
QQYLNQSHVRVLELPFQMSEFYVGLHWFAQTNEPLARIWLIQTCKKVISVL*
or at least 90%, 95%, 96%, 97%, 98%, 99%
or 100% identity thereto.

Accession number: WP_343682615.1.

### III. Plasmid Retention Mechanisms

Plasmid retention mechanisms are strategies used to ensure that plasmids, which are circular DNA molecules separate from chromosomal DNA, are maintained within bacterial cells during cell division. These mechanisms are needed in biotechnology and genetic engineering, where plasmids often carry genes of interest, such as those encoding therapeutic proteins or other beneficial traits.

Provided below are several plasmid retention mechanisms:

Antibiotic Resistance Markers: Plasmids often carry genes that confer resistance to specific antibiotics. When bacteria are grown in the presence of these antibiotics, only those cells that retain the plasmid survive, as they can resist the antibiotic's effects.

Auxotrophic Markers: This involves using a plasmid to complement a genetic deficiency in the host cell. For example, if the host cell lacks the ability to synthesize a particular amino acid, the plasmid can carry a gene that restores this ability. The host cell must retain the plasmid to survive in media lacking that amino acid.

Toxin-Antitoxin Systems: These systems involve a pair of genes on the plasmid: one encoding a stable toxin and the other encoding an unstable antitoxin. If the plasmid is lost, the antitoxin degrades faster than the toxin, leading to cell death.

Partitioning Systems: These systems ensure that plasmids are evenly distributed between daughter cells during cell division. They often involve proteins that actively segregate plasmids to opposite poles of the cell before division.

Plasmid Addiction Systems: Similar to toxin-antitoxin systems, these systems make the host cell dependent on the plasmid for survival. The plasmid encodes a function that is essential for the host cell's survival, making it “addicted” to the plasmid.

In one embodiment, the plasmid retention mechanism involves a genomic asd (NT sequence:

(SEQ ID NO: 58)
Atgaaaaatgttggttttatcggctggcgcggaatggtcggctctg
ttctcatgcaacgcatggtagaggagcgcgatttcgacgctattc
gccctgttttcttttctacctcccagtttggacaggggcgcccac
cttcggcgacacctccggcacgctacaggacgcttttgacctgga
tgcgctaaaagcgctcgatatcatcgtgacctgccagggcggcga
ttataccaacgaaatttatccaaagctgcgcgaaagcggatggca
gggttactggattgatgcggcttctacgctgcgcatgaaagatga
tgccattattattctcgacccggtcaaccaggacgtgattaccga
cggcctgaacaatggcgtgaaaacctttgtgggcggtaactgtac
cgttagcctgatgttgatgtcgctgggcggtctgtttgcccataa
tctcgttgactgggtatccgttgcaacttatcaagcagcttctgg
tggtggtgcacgtcatatgcgtgagctgttaacccagatgggtca
gttgtatggccatgtcgccgatgaactggcgacgccgagctccgc
aattcttgatattgaacgcaaagttacggcattgacccgcagcgg
cgagctgccggttgataactttggcgtaccgctggcgggaagcct
gatcccctggatcgacaaacagctcgataacggccagagccgcga
agagtggaaaggccaggcggaaaccaacaagattctcaatactgc
ctctgtgattccggttgatggtttgtgtgtgcgcgtcggcgcgct
gcgctgtcacagccaggcgttcaccatcaagctgaaaaaagaggt
atccattccgacggtggaagaactgctggcggcacataatccgtg
ggcgaaagtggtgccgaacgatcgtgatatcactatgcgcgaatt
aaccccggcggcggtgaccggcacgttgactacgccggttggtcg
tctgcgtaagctgaacatggggccagagttc
or at least 90%, 95%, 96%, 97%,
98%, 99% or 100% identity
thereto.

AA sequence: MVKDAPQDTGAHTQHISLQEKNAMKNVGFIGWRGMVGSVLMQRMVEERDFDAIRPVF FSTSQFGQAAPTFGDTSGTLQDAFDLDALKALDIIVTCQGGDYTNEIYPKLRESGWQGY WIDAASTLRMKDDAIIILDPVNQDVITDGLNNGVKTFVGGNCTVSLMLMSLGGLFAHNL VDWVSVATYQAASGGGARHMRELLTQMGQLYGHVADELATPSSAILDIERKVTALTRSG ELPVDNFGVPLAGSLIPWIDKQLDNGQSREEWKGQAETNKILNTASVIPVDGLCVRVGA LRCHSQAFTIKLKKEVSIPTVEELLAAHNPWAKVVPNDRDITMRELTPAAVTGTLTTPVG RLRKLNMGPEFLSAFTVGDQLLWGAAEPLRRMLRQLA* (SEQ ID NO: 59) or at least 90%, 95%, 96%, 97%, 98%, 99% or 100% identity thereto. (Accession number: AAB69392.1) knockout combined with the expression of pAsd-asd on the plasmid. This means that the host bacteria are engineered to lack the asd gene, which is needed for cell wall synthesis. The plasmid provides the asd gene, so the bacteria must retain the plasmid to survive, as it supplies the necessary function for cell viability.

### IV. Therapeutic Proteins/Peptides

In some embodiments the therapeutic protein to be expressed by the bacterial cells provided herein are globular proteins. Globular proteins are compact, spherical proteins that are often used in medical treatments due to their specific biological activities. Some examples include, but are not limited to, cytokines, such as interferons (e.g., IFN-γ (nucleotide sequence: Atggattataaagatgatgatgataaaatgaaatataccagctatattctggcgtttcctgtgcattgtgctgggcagcctgggctgctattgcc aggatccgtatgtgaaagaagcggaaaacctgaaaaaatattttaacgcgggccatagcgatgtggcggataacggcaccctgtttctggg cattctgaaaaactggaaagaagaaagcgatcgcaaaattatgcagagccagattgtgagcttttattttaaactgtttaaaaactttaaagatg atcagagcattcagaaaagcgtggaaaccattaaagaagatatgaacgtgaaattttttaacagcaacaaaaaaaaacgcgatgattttgaaa aactgaccaactatagcgtgaccgatctgaacgtgcagcgcaaagcgattcatgaactgattcaggtgatggcggaactgagcccggcgg cgaaaaccggcaaacgcaaacgcagccagatgctgtttegcggccgccgcgcgagccagTAA; ((SEQ ID NO: 60) or at least 90%, 95%, 96%, 97%, 98%, 99% or 100% identity thereto) amino acid sequence: MDYKDDDDKMKYTSYILAFQLCIVLGSLGCYCQDPYVKEAENLKKYFNAGHSDVADN GTLFLGILKNWKEESDRKIMQSQIVSFYFKLFKNFKDDQSIQKSVETIKEDMNVKFFNSN KKKRDDFEKLTNYSVTDLNVQRKAIHELIQVMAELSPAAKTGKRKRSQMLFRGRRASQ (SEQ ID NO: 61) or at least 90%, 95%, 96%, 97%, 98%, 99% or 100% identity thereto. (Accession number: NP_000610.2) (these are cytokines used in the treatment of various diseases, including certain cancers and viral infections); insulin (a hormone used to regulate blood sugar levels in individuals with diabetes); erythropoietin (EPO) (a hormone that stimulates red blood cell production, used to treat anemia, particularly in chronic kidney disease); growth hormone (GH) (used to treat growth disorders in children and growth hormone deficiency in adults); monoclonal antibodies (e.g., Trastuzumab, Rituximab) (these are engineered antibodies used to target specific antigens, commonly used in cancer therapy and autoimmune diseases); ScFvs; shark/camelid nanobodies; enzymes (e.g., Tissue Plasminogen Activator, TPA) (such as those used to dissolve blood clots in the treatment of stroke and heart attack); and/or tumor necrosis factor (TNF) inhibitors (e.g., Infliximab) (used to treat autoimmune diseases such as rheumatoid arthritis and Crohn's disease).

These proteins are typically used for their ability to interact with specific biological targets, making them effective in treating a wide range of conditions.

### V. Cancer Treatment

Bacteria such as Salmonella, Clostridium and Bifidobacterium have a natural tropism for cancers, such as solid tumors. Types of cancer that can be treated using the methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some aspects, the subject is treated with radiation and chemotherapy before, after or during administration of the bacterial cells described herein.

### VI. Administration

The invention includes administration of the bacteria, such as attenuated Salmonella strains described herein, and methods for preparing pharmaceutical compositions and administering such as well. Such methods comprise formulating a pharmaceutically acceptable carrier with one or more of the bacterial strains described herein.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intravenous administration, suitable carriers include physiological saline, bacteriostatic water, Cremophor EL™ (BASF; Parsippany, N.J.) or phosphate buffered saline (PBS). It must be stable under the conditions of manufacture and storage and must be preserved against the contaminating action of other (undesired) microorganisms. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, the preferred methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the bacteria are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

When administered to a patient the attenuated Salmonella can be used alone or may be combined with any physiological carrier. In general, the dosage ranges from about 1.0 c.f.u./kg to about 1×10¹² c.f.u./kg; optionally from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg; optionally from about 1×10⁵ c.f.u./kg to about 1×10¹² c.f.u./kg; optionally from about 1×10⁵ c.f.u./kg to about 1×10¹⁰ c.f.u./kg; optionally from about 1×10⁵ c.f.u./kg to about 1×10⁸ c.f.u./kg.

### EXAMPLES

The following examples are provided in order to demonstrate and further illustrate certain embodiments and aspects of the present invention and are not to be construed as limiting the scope thereof.

Salmonella colonize tumors 10,000-fold more than any other healthy organ. The bacteria are facultative anaerobes that can target viable, as well as hypoxic tumor regions. The bacteria colonize and only grow exponentially within tumors due to the immune-privileged microenvironment, which limits microbial clearance. Tumor specific growth of Salmonella allow the bacteria to deliver 1,000- to 10,000-fold more therapy in tumors than the originally administered dose. Thus, engineering Salmonella to deliver therapies that act extracellularly is ideal for increasing tumor efficacy without causing off-target toxicity.

Provided herein are engineered Salmonella that selectively colonize, lyse and deliver therapies extracellularly within the tumor microenvironment. The Salmonella were engineered with an flhD knockout (to keep bacteria from invading cancer cells and in the extracellular space), asd knockout (for plasmid retention) and sseJ knockout (for attenuation). In addition, the bacteria were engineered to lyse with an over-the-counter dose of aspirin and release a protein-based therapy extracellularly.

As an example, one of these extracellularly delivered cytokines is IFNγ. IFNγ is a cytokine that plays a role in inhibiting tumor growth. It converts the tumor microenvironment from immunosuppressive to immunostimulatory 1. It does so by converting the macrophages from M2 to M1 phenotype1,2. It also causes apoptosis of the regulatory T cells1 and activation of CD8 T cells and Tₕ1 CD4 T cells2-4. Further it upregulates MHC I on cancer cells which are inherently downregulated by cancer cells to evade immune system2,5. Moreover, it has anti-proliferative effects causing cell cycle arrest or apoptosis of cancer cells2,4. It also prevents angiogenesis which further inhibits tumor growth and metastases1,2. Despite playing a multifunctional role in tumor suppression, IV delivery still poses a problem.

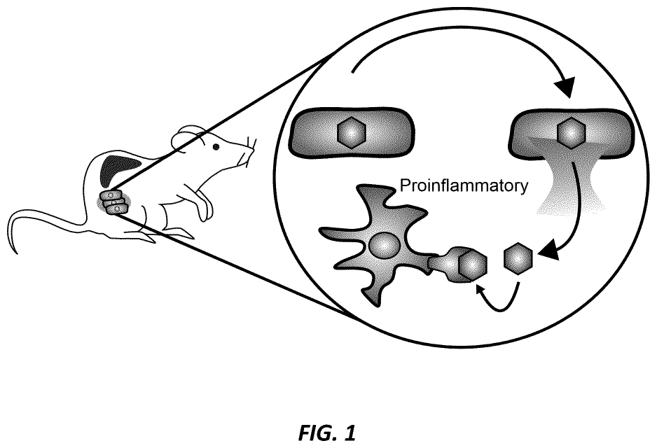

IFNγ when injected intravenously in blood has a half-life of 30 minutes and thus gets cleared out very quickly before even making to the tumor microenvironment6. This short half-life requires a large dose of IFNγ which causes severe side effects including neurotoxicity and leukopenia7. Acute IFNγ has a tumor suppressive role whereas chronic IFNγ can be tumor promoting. Chronic IFNγ can cause PD-L1 upregulation on cancer cells7. To provide a solution to all the problem, an extracellular delivering Salmonella (ED Sal) was created that does not invade cancer cell and lyse extracellularly to deliver IFNγ providing an acute dose of IFNγ (FIG. 1).

### Example I

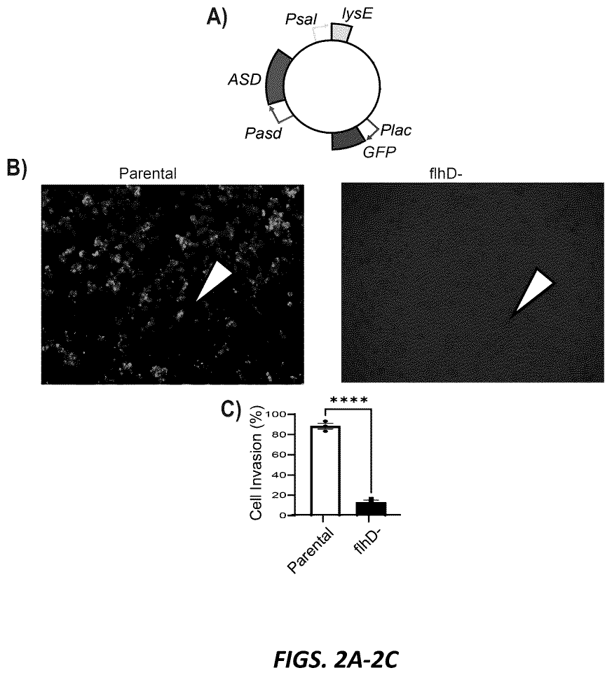

Salmonella strain lacking in flhD was incapable of invading cells: Salmonella having a knockout of the flhD gene and transformed with a plasmid with green florescence protein (GFP; FIG. 2A) being expressed constitutively was co-cultured with mouse pancreatic cancer cells (KPCY) at a Multiplicity of infection (MOI) of 20 with Parental Salmonella (VNP20009) transformed with same plasmid as a control. 2 hours post infections cells were washed with PBS 3× times. Fresh media with 50 mg/ml of gentamycin was added to cells to kill all the extracellular bacteria. Two hours post that cells were fixed with formaldehyde and imaged. Little over 10% of flhD-Salmonella were present inside the cells while over 85% of the parental Salmonella were inside cells (FIGS. 2B and C) (P<0.0001). flhD-strain was then transformed with a plasmid that had the lysE gene under the Psal promoter to facilitate extracellular lysis and deliver IFNγ when induced with salicylic acid is termed as extracellular delivering Salmonella (ED Sal).

### Example II

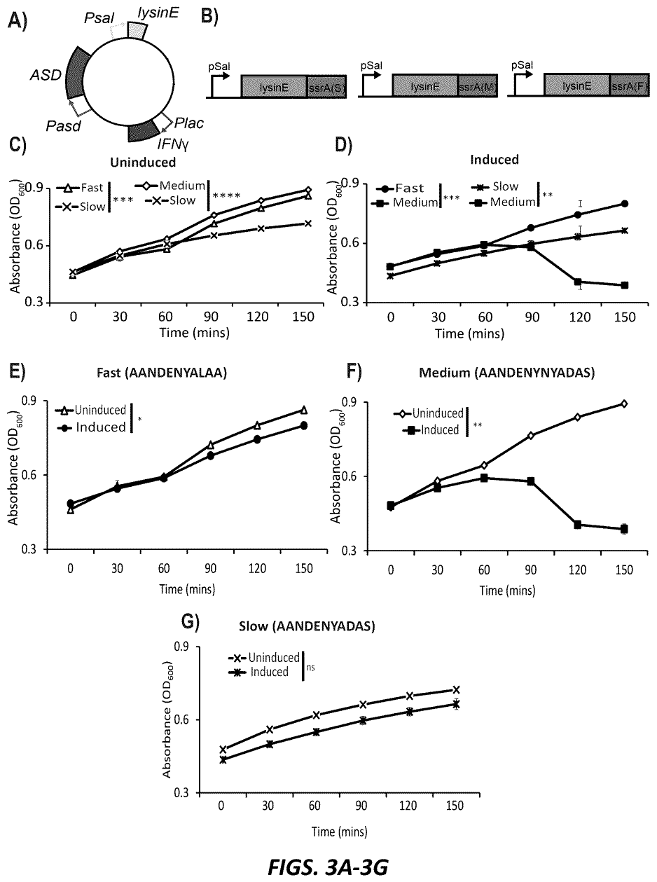

Tuning of SSRA degradation tag is needed for effective lysis: In order to create an IFNγ extracellular delivery (ED) strain, the non-invasive Salmonella strain (FIG. 2) was transformed with a plasmid containing the ASD, placUV5-IFNγ and one of three expression-tuned, pSal-lysin E genetic constructs (FIG. 3A): ED Sal-IFNγ-slow, ED Sal-IFNγ-medium, or ED Sal-IFNγ-fast (FIG. 3B). The three different degradation tags adjust protein expression levels such that higher degradation rates correspond to lower expression levels. In the absence of salicylic acid (the active metabolic product of Aspirin), all three strains of ED Sal-IFNγ continued to grow in broth culture (FIG. 3C). Two-and-a-half hours after induction with salicylic acid, only the ED Sal-IFNγ-medium strain lysed, as indicated by a significant reduction in bacterial density (FIG. 3D). Bacterial growth remained largely unchanged for both ED Sal-IFNγ-fast (FIG. 3E) and ED Sal-IFNγ-slow (FIG. 3F) after salicylic acid induction. However, a significant fraction of ED Sal-IFNγ-medium lysed after induction with salicylic acid. These results indicate that only lysin E containing a medium strength, C-terminal ssrA tag was needed to induce bacterial lysis after induction of salicylic acid.

### Example III

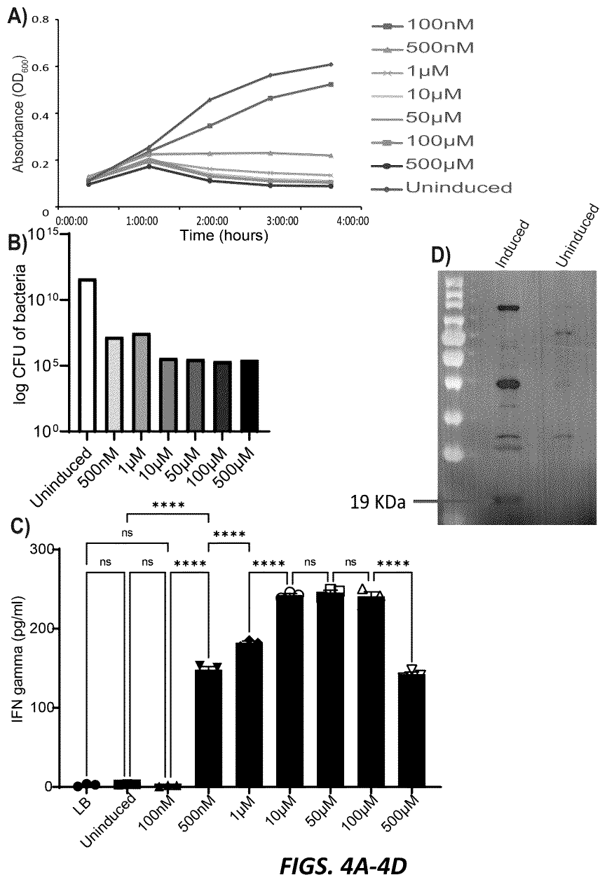

Induction with salicylic acid causes ED Sal-IFNγ to lyse and release IFNγ: Salicylic acid was added to the culture of ED Sal-IFNγ at different concentrations (100 nM, 500 nM, 1 μM, 10 μM, 50 μM, 100 μM, and 500 μM) where uninduced was used a control. Except for 100 nM every other concentration was able to cause the Salmonella to lyse (FIG. 4A). 4 hours after induction there were approximately 10⁷ CFU of bacteria in the culture induced with 500 nM and 1 μM and approximately 10⁵ CFU of bacteria in culture induced with 10 μM, 50 μM, 100 μM, and 500 μM (FIG. 4B). Over 10¹¹ CFU of bacteria were present in the uninduced culture (FIG. 4B). The induction caused 99.99% of the bacteria to lyse and release IFNγ in the lysate. The concentration of IFNγ increased with the increasing concentration of Salicylic acid until 10 μM and then stayed constant until 100 μM and then further decreased for 500 μM (FIG. 4C) (P<0.0001). ED Sal-IFNγ culture when induced with 500 μM released full length IFNγ as seen on the western blot. (FIG. 4D).

### Example IV

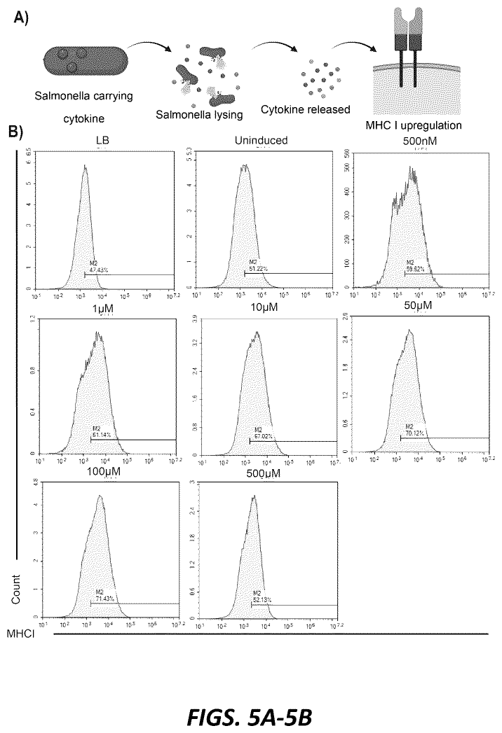

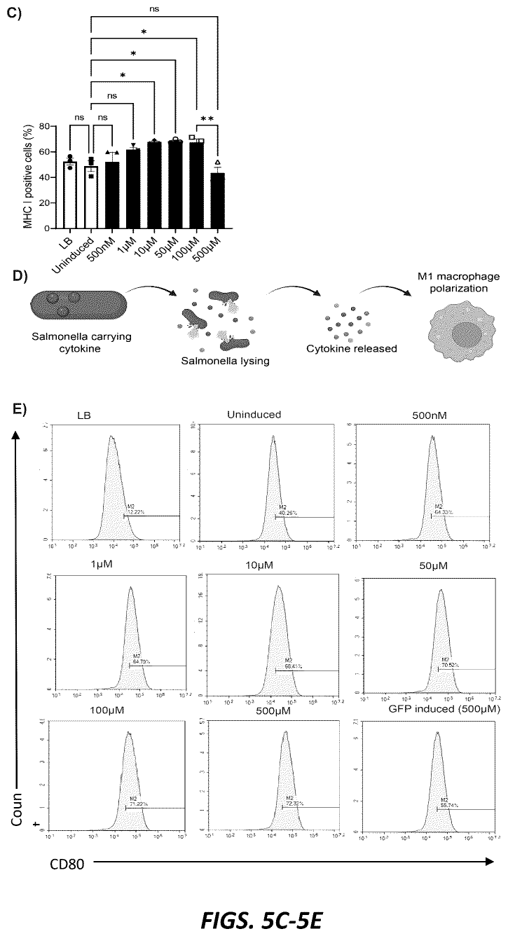

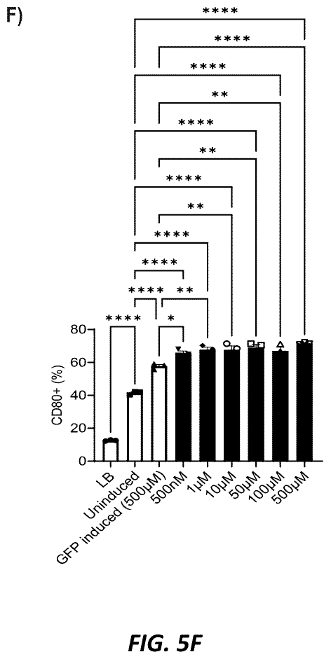

IFNγ released from ED Sal upregulates MHC I expression on Melanoma cells and polarization macrophages to M1 phenotype: ED Sal-IFNγ when induced with salicylic acid caused the bacteria to lyse and release IFNγ. The lysate was then put on mouse melanoma cells (B16F10) (FIG. 5A) where the control was the uninduced bacteria. The lysate (10 μM, 50 μM, and 100 μM) containing IFNγ significantly upregulated MHC I expression on cells (FIGS. 5B and C) (P<0.05). There was a significant reduction in the MHC I expression when induced with 500 μM Salicylic acid which was consistent with the IFNγ released (FIG. 5C) (P<0.01). The lysate containing the IFNγ was put on macrophages (RAW 264.7 cells) to see whether this cytokine polarized them to a M1 phenotype (FIG. 5D). Here, beside uninduced bacteria, ED Sal-GFP was also used a control as bacterial lysate can polarize macrophages. There was a significant increase in the polarization of macrophages into M1 phenotype as compared to uninduced and ED Sal-GFP (FIGS. 5E and F) (P<0.5, P<0.01, P<0.0001).

### Example V

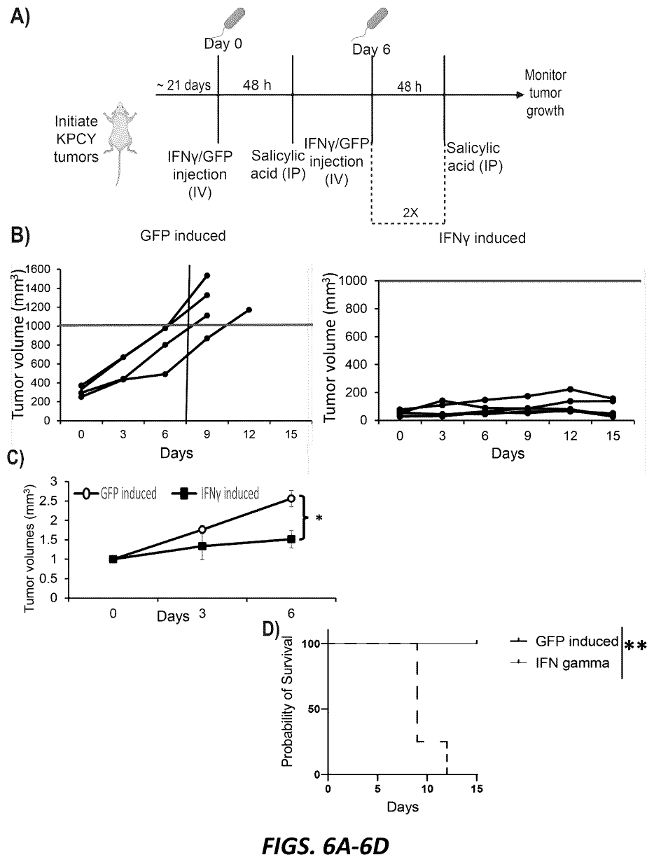

Delivery of IFNγ in mice implanted with KPCY tumors decreases tumor volumes and increases survival: C57BL/6 mice implanted with KPCY tumors were injected intravenously (IV) with 1*10⁷ CFU of ED Sal-IFNγ, with ED Sal-GFP as a control. Two days after the injection 50 mg/ml of Salicylic acid was injected intraperitonially (IP). This procedure was repeated two more times and tumors were measured every three days until maximum tumor burden was reached (1000 mm³) (FIG. 6A). The delivery of IFNγ slowed down the tumor growth as compared to the control, where the first control died on day 9 whereas for mice injected with IFNγ 100% of the mice survived past day 9 (FIG. 6B). There was over 40% reduction in tumor volume of the mice in the experimental group by day 6 (P<0.05) (FIG. 6C). This delivery also significantly increased the survival of the experimental group (P<0.01) (FIG. 6D).

### Example VI

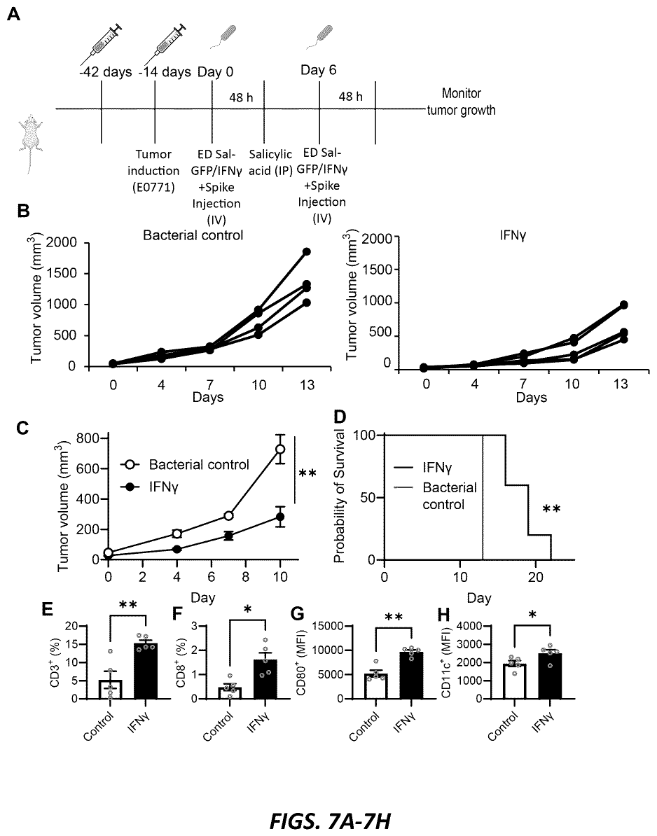

Delivering IFNγ extracellularly and spike protein intracellularly to increase antigen presentation of spike: Spike when administered IT has a precedence of decreasing tumor volumes and increasing overall survival. Here, it was determined whether administration of IFNγ along with Spike can be delivered IV and cause reduction in tumor volumes and increase overall survival. IFNγ upregulates MHC I and will enhance presentation of spike antigen. Mice were immunized with the spike vaccine from Pfizer at day 0, 28 days later a booster was given to the mice and implanted with mouse breast cancer (E0771) cells. When tumors were between 15-40 mm³, 2.5*106 CFU each of ED Sal-IFNγ and Spike plasmid transformed in the parental strain were injected IV. Two days after injection 50 mg/ml of Salicylic acid was given IP. The entire procedure was repeated one more time (FIG. 6A). For control, Spike was injected with ED Sal-GFP and tumors were measured every three days for efficacy and survival studies until the maximum tumor burden is reached (1000 mm³) (FIG. 7B). There was over 60% reduction in tumor volume of the mice in the experimental group by day 6 (P=0.0097) (FIG. 7C). This delivery also significantly increased the survival of the experimental group (P=0.0047) (FIG. 7D). In addition, intratumoral delivery of IFNγ and spike increased the number of i) infiltrated CD3 T cells (P=0.0098, FIG. 7E); ii) CD8+ T cells (P=0.0109, FIG. 7F); iii) CD80+ activated macrophages (P=0.0010, FIG. 7G); and iv) CD11c+ dendritic cells (P=0.0486, FIG. 7H).

### Example VII

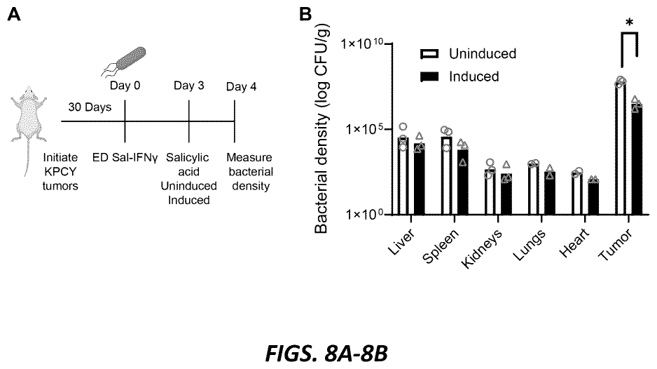

Activation of the Salicylic Acid responsive lysis circuit reduces intratumoral bacterial load: Salicylic-acid induction markedly reduced the intratumoral bacterial burden in mice bearing KPCY tumors (FIG. 8). C57BL/6 mice were injected intravenously with 1*10⁷ CFU of ED Sal-IFN strain, and tumors were allowed to accumulate bacteria for three days. At that point, one cohort received an intraperitoneal injection of salicylic acid (50 mg/mL) to activate the lysis circuit, while the control cohort remained uninduced. Twenty-four hours later, all mice were euthanized and bacterial loads were quantified across major organs, including liver, spleen, kidneys, lungs, heart, and tumor (FIG. 8A). Salicylic-acid induction led to a modest reduction in bacterial density across healthy organs, with decreases ranging from 1.5-fold to 5.5-fold. The most pronounced effect occurred in tumors, where induction triggered a 17-fold decrease in viable bacterial counts compared with uninduced controls (FIG. 8B, P=0.030). This sharp reduction in tumor bacterial burden is consistent with circuit driven lysis of the engineered strain following salicylic acid activation and supports the conclusion that bacteria lysed within the tumor microenvironment

### Discussion

ED Sal-IFNγ successfully lysed after salicylic acid induction. IFNγ release upregulates MHC I, polarizes macrophages in vitro and reduced tumor volumes in vivo. Salmonella having a deletion in the flhD gene do not invade cells when compared to parental Salmonella, where most of the Salmonella were inside cancer cells. ED Sal-IFNγ when induced with different concentrations of salicylic acid lyse where there is no difference in the lysis after 500 nM of salicylic acid. After lysis these Salmonella release IFNγ in the lysate. The lysate when put on B16F10s cell upregulate MHC I as compared to the uninduced bacteria. The IFNγ containing lysate also polarizes macrophages to M1 phenotype when compared to ED Sal-GFP. IV delivery of ED Sal-IFNγ causes 40% reduction in tumor volumes in 6 days and prolonged survival in vivo when compared to ED Sal-GFP.

### BIBLIOGRAPHY

1 Jorgovanovic, D., Song, M., Wang, L., and Zhang, Y. (2020). Roles of IFN-gamma in tumor progression and regression: a review. Biomark Res 8, 49. 10.1186/s40364-020-00228-x.
2. Mojic, M., Takeda, K., and Hayakawa, Y. (2017). The Dark Side of IFN-gamma: Its Role in Promoting Cancer Immunoevasion. Int J Mol Sci 19. 10.3390/ijms19010089.
3. Muhl, H., and Pfeilschifter, J. (2003). Anti-inflammatory properties of pro-inflammatory interferon-gamma. Int Immunopharmacol 3, 1247-1255. 10.1016/S1567-5769(03)00131-0.
4. Chen, Y., Du, M., Yuan, Z., Chen, Z., and Yan, F. (2024). Author Correction: Spatiotemporal control of engineered bacteria to express interferon-gamma by focused ultrasound for tumor immunotherapy. Nat Commun 15, 3656. 10.1038/s41467-024-48061-2.
5. Zhou, F. (2009). Molecular mechanisms of IFN-gamma to up-regulate MHC class I antigen processing and presentation. Int Rev Immunol 28, 239-260. 10.1080/08830180902978120.
6. Ando, M., Takahashi, Y., Yamashita, T., Fujimoto, M., Nishikawa, M., Watanabe, Y., and Takakura, Y. (2014). Prevention of adverse events of interferon gamma gene therapy by gene delivery of interferon gamma-heparin-binding domain fusion protein in mice. Mol Ther Methods Clin Dev 1, 14023. 10.1038/mtm.2014.23.
7. Zhang, X., Wang, S., Zhu, Y., Zhang, M., Zhao, Y., Yan, Z., Wang, Q., and Li, X. (2021). Double-edged effects of interferons on the regulation of cancer-immunity cycle. Oncoimmunology 10, 1929005. 10.1080/2162402X.2021.1929005.
Gurbatri C R, Lia I, Vincent R, Coker C, Castro S, Treuting P M, Hinchliffe T E, Arpaia N, Danino T. Engineered probiotics for local tumor delivery of checkpoint blockade nanobodies. Sci Transl Med. 2020 Feb. 12; 12(530):eaax0876. doi: 10.1126/scitranslmed.aax0876. PMID: 32051224; PMCID: PMC7685004.
Chowdhury S, Castro S, Coker C, Hinchliffe T E, Arpaia N, Danino T. Programmable bacteria induce durable tumor regression and systemic antitumor immunity. Nat Med. 2019 July; 25(7):1057-1063. doi: 10.1038/s41591-019-0498-z. Epub 2019 Jul. 3. PMID: 31270504; PMCID: PMC6688650.
Danino T, Mondragón-Palomino O, Tsimring L, Hasty J. A synchronized quorum of genetic clocks. Nature. 2010 Jan. 21; 463(7279):326-30. doi: 10.1038/nature08753. PMID: 20090747; PMCID: PMC2838179.
Li F, Yang Z, Savage T M, Vincent R L, de Los Santos-Alexis K, Ahn A, Rouanne M, Mariuzza D L, Danino T, Arpaia N. Programmable bacteria synergize with PD-1 blockade to overcome cancer cell-intrinsic immune resistance mechanisms. Sci Immunol. 2024 Oct. 18; 9(100):eadn9879. doi: 10.1126/sciimmunol.adn9879. Epub 2024 Oct. 18. PMID: 39423284.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A bacteria comprising a plasmid comprising a nucleic acid sequence coding for a therapeutic protein, a salicylic acid inducible promotor operably linked to a nucleic acid sequence coding for a lysis protein and a plasmid retention mechanism.

2. The bacteria of claim 1, wherein the bacteria do not intracellularly invade cancer cells.

3. The bacteria of claim 1, wherein the bacteria are E. coli or Salmonella.

4. The bacteria of claim 3, wherein the E. coli is E. coli Nissle or MG1655.

5. The bacteria of claim 3, wherein the Salmonella is Salmonella typhimurium.

6. The bacteria of claim 3, wherein the Salmonella is non-motile.

7. The bacteria of claim 3, wherein the Salmonella contains an flhD, fliC, flgE or an fliGHI knockout or any combination thereof.

8. The bacteria of claim 1, wherein the salicylic acid inducible promoter is from Acinetobacter baylyi.

9. The bacteria of claim 1, wherein the lysis protein is lysin gene E from phage phiX174.

10. The bacteria of claim 9, wherein the lysin gene E contains a C-terminal ssrA degradation sequence.

11. The bacteria of claim 10, wherein the C-terminal ssrA degradation sequence comprises or consists of AANDENYALAA (SEQ ID NO: 53).

12. The bacteria of claim 10, wherein the C-terminal ssrA degradation sequence comprises or consists of AANDENYADAS (SEQ ID NO: 54).

13. The bacteria of claim 10, wherein the C-terminal ssrA degradation sequence comprises or consists of AANDENYNYADAS (SEQ ID NO: 55).

14. The bacteria of claim 1, wherein the therapeutic protein is a globular protein.

15. The bacteria of claim 14, wherein the globular protein is between 10 and 30 kilodaltons.

16. The bacteria of claim 14, wherein the globular protein is IFNγ.

17. The bacteria of claim 1, wherein the plasmid retention mechanism is a genomic asd knockout combined with pAsd-asd expressed on the plasmid.

18. A method to treat cancer comprising administering the bacteria of claim 1 to subject in need thereof.

19. The method of claim 18, wherein the subject is human.

20. The method of claim 18, wherein the cancer comprises a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma or retinoblastoma.


SEQUENCE LISTING (61 sequences)

SEQ ID NO: 1 - protein, 400 aa, Salmonella typhimurium
MPLSVGQGYFTSSISSEKFNAIKESARLPELSLWEKIKAYFFTTHHAEALECIFNLYHHQELNLTPVQVRGAYIKLRALASQGCKEQFIIESQEHADKLIIKDDNGENILSIEVECHPEAFGLAKEINKSHPKPKNISLGDITRLVFFGDSLSDSLGRMFEKTHHILPSYGQYFGGRFTNGFTWTEFLSSPHFLGKEMLNFAEGGSTSASYSCFNCIGDFVSNTDRQVASYTPSHQDLAIFLLGANDYMTLHKDNVIMVVEQQIDDIEKIISGGVNNVLVMGIPDLSLTPYGKHSDEKRKLKDESIAHNALLKTNVEELKEKYPQHKICYYETADAFKVIMEAASNIGYDTENPYTHHGYVHVPGAKDPQLDICPQYVFNDLVHPTQEVHHCFAIMLESF

SEQ ID NO: 2 - DNA, 1227 bp, Salmonella typhimurium
  source 1..1227: mol_type = other DNA
atgccattgagtgttggacagggttatttcacatcatctatcagttctgaaaaatttaatgcgataaaagaaagcgcacgccttccggaattaagtttatgggagaaaatcaaagcatatttctttaccacccaccatgcagaggcgctcgaatgtatctttaatctttaccaccatcaggaactgaatctaacaccggtacaggttcgcggagcctacatcaaacttcgagccttagcgtctcagggatgtaaagaacagtttattatagaatcacaggaacacgccgataagttgattattaaagatgataatggtgaaaatattttgtctattgaggttgaatgtcatccggaagcttttggtcttgcaaaagaaatcaataaatcacatcccaagcccaaaaatatttctttgggtgatattaccagactggtattttttggcgacagcttgtctgactccttagggcgtatgtttgaaaaaacacatcatatcttaccctcctatggtcaatactttggcggaaggtttactaatggatttacctggactgagtttttatcatctccacacttcttaggtaaagagatgcttaattttgctgaagggggaagtacatcggcaagctattcctgctttaattgcatcggtgactttgtatcaaatacggacagacaagtcgcatcttacaccccttctcaccaggacctggcgatatttttattgggggctaatgactatatgacactacacaaagataatgtaataatggtcgttgagcaacaaattgatgatattgaaaaaataatttccggtggagttaataatgttctggtcatggggattcccgatttgtctttaacaccttatggcaaacattctgatgaaaaaagaaagcttaaggatgaaagcatcgctcacaatgccctgttaaaaactaatgttgaagaattaaaagaaaaatacccccagcataaaatatgctattacgagactgccgatgcatttaaggtgataatggaggcggccagtaatattggttatgatacggaaaacccttatactcaccacggctatgtacatgttcccggggctaaagaccctcagctagatatatgtccgcaatacgtcttcaacgaccttgtccatccaacccaggaagtccatcattgttttgccataatgttagaaagttttatagctcatcattattccactgaataa

SEQ ID NO: 3 - DNA, 923 bp, Salmonella typhimurium
  source 1..923: mol_type = other DNA
atgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 4 - protein, 113 aa, Salmonella typhimurium
MHTSELLKHIYDINLSYLLLAQRLIVQDKASAMFRLGINEEMANTLGALTLPQMVKLAETNQLVCHFRFDDHQTITRLTQDSRVDDLQQIHTGIMLSTRLLNEVDDTARKKRA

SEQ ID NO: 5 - protein, 192 aa, Salmonella typhimurium
MSEKSIVQEARDIQLAMELINLGARLQMLESETQLSRGRLIRLYKELRGSPPPKGMLPFSTDWFMTWEQNIHASMFCNAWQFLLKTGLCSGVDAVIKAYRLYLEQCPQPPEGPLLALTRAWTLVRFVESGLLELSSCNCCGGNFITHAHQPVGSFACSLCQPPSRAVKRRKLSRDAADIIPQLLDEQIEQAV

SEQ ID NO: 6 - protein, 295 aa, Salmonella typhimurium
MLILLGYLVVIGTVFGGYVMTGGHLGALYQPAELVIIGGAGIGAFIVGNNGKAIKGTMKAIPLLFRRSKYTKSMYMDLLALLYRLMAKSRQQGMFSLERDIENPKESEIFASYPRILADAVMLDFIVDYLRLIISGNMNTFEIEALMDEEIETHESEAEVPANSLAMVGDSLPAFGIVAAVMGVVHALASADRPAAELGALIAHAMVGTFLGILLAYGFISPLATVLRQKSAETTKMMQCVKITLLSNLNGYAPPIAVEFGRKTLYSSERPSFIELEEHVRAVRNPNQQQTTEEA

SEQ ID NO: 7 - protein, 309 aa, Salmonella typhimurium
MKNQAHPIVVVKRRRHKPHGGGAHGSWKIAYADFMTAMMAFFLVMWLISISSPKELIQIAEYFRTPLATAVTGGNRIANSESPIPGGGDDYTQQQGEVEKQPNIDELKKRMEQSRLNKLRGDLDQLIESDPKLRALRPHLKIDLVQEGLRIQIIDSQNRPMFKTGSAEVEPYMRDILRAIAPVLNGIPNRISLAGHTDDFPYANGEKGYSNWELSADRANASRRELVAGGLDNGKVLRVVGMAATMRLSDRGPDDAINRRISLLVLNKQAEQAILHENAESQNEBVSVLQQPAAAPPASVPTSPKAEPR

SEQ ID NO: 8 - protein, 130 aa, Salmonella typhimurium
MRKWLALLLFPLTVQAAGEGAWQDSGMGVTLNYRGVSASSSPLSARQPVSGVMTLVAWRYELNGPTPAGLRVRLCSQSRCVELDGQSGTTHGFAHVPAVEPLRFVWEVPGGGRLIPALKVRSNQVIVNYR

SEQ ID NO: 9 - protein, 214 aa, Salmonella typhimurium
MMQPSIKPADEGSAGDIIARIGSLTRMLRDSLRELGLDQAIAEAAEAIPDARDRLDYVVQMTAQAAERALNSVEASQPHQDAMEKEAKALTQRWDEWFDNPIELSDARELVTDTRQFLRDVPGHTSFTNAQLLDIMMAQDFQDLTGQVIKRMMDVIQEIERQLLMVLLENIPEQSARPKRENESLLNGPQVDTSKAGVVASQDQVDDLLDSLGF

SEQ ID NO: 10 - protein, 129 aa, Salmonella typhimurium
MADKELKFLVVDDFSTMRRIVRNLLKELGFNNVEEAEDGVDALNKLQAGGFGFIISDWNMPNMDGLELLKTIRADSAMSALPVLMVTAEAKKENIIAAAQAGASGYVVKPFTAATLEEKLNKIFEKLGM

SEQ ID NO: 11 - protein, 349 aa, Salmonella typhimurium
MSKIRVLSVDDSALMRQIMTEIINSHSDMEMVATAPDPLVARDLIKKFNPDVLTLDVEMPRMDGLDFLEKLMRLRPMPVVMVSSLTGKGSEVTLRALELGAIDFVTKPQLGIREGMLAYSEMIAEKVRTAARARIAAHKPMAAPTTLKAGPLLSSEKLIAIGASTGGTEAIRHVLQPLPLSSPAVIITQHMPPGFTRSFAERLNKLCQISVKEAEDGERVLPGHAYIAPGDKHMELARSGANYQIKIHDGPPVNRHRPSVDVLFHSVAKHAGRNAVGVILTGMGNDGAAGMLAMYQAGAWTIAQNEASCVVFGMPREAINMGGVSEVVDLSQVSQQMLAKISAGQAIRI

SEQ ID NO: 12 - protein, 288 aa, Salmonella typhimurium
MTSSLPSGQTSVLLQMTQRLALSDAHFRRICQLIYQRAGIVLADHKRDMVYNRLVRRLRALGLDDFGRYLSMLEANQNSAEWQAFINALTTNLTAFFREAHHFPILAEHARRRHGEYRVWSAAASTGEEPYSIAITLADALGMAPGRWKVFASDIDTEVLEKARSGIYRLSELKTLSPQQLQRYFMRGTGPHEGLVRVRQELANYVEFSSVNLLEKQYNVPGPFDAIFCRNVMIYFDKTTQEDILRRFVPLLKPDGLLFAGHSENFSNLVREFSLRGQTVYALSKDKA

SEQ ID NO: 13 - protein, 553 aa, Salmonella typhimurium
MFNRIRVVTMLMMVLGVFALLQLVSGGLLFSSLQHNQQGFVISNELRQQQSELTSTWDLMLQTRINLSRSAARMMMDASNQQSSAKTDLLQNAKTTLAQAAAHYANFKNMTPLPAMAEASANVDEKYQRYQAALAELIQFLDNGNMDAYFAQPTQGMQNALGEALGNYARVSENLYRQTFDQSAHDYRFAQWQLGVLAVVLVLILMVVWFGIRHALLNPLARVITHIREIASGDLTKTLTVSGRNEIGELAGTVEHMQRSLIDTVTQVREGSDAIYSGTSEIAAGNTDLSSRTEQQASALEETAASMEQLTATVKQNADNARQASQLAQSASETARHGGKVVDGVVNTMHEIADSSKKIADIISVIDGIAFQTNILALNAAVEAARAGEQGRGFAVVAGEVRNLASRSAQAAKEIKALIEDSVSRVDTGSVLVESAGETMTDIVNAVTRVTDIMGEIASASDEQSRGIDQVALAVSEMDRVTQQNASLVQESAAAAAALEEQASRLTQAVSAFRLASRPLAVNKPEMRLSVNAQSGNTPQSLAARDDANWETF

SEQ ID NO: 14 - protein, 167 aa, Salmonella typhimurium
MTGMSNVSKLAGEPSGQEFLVFTLGNEEYGIDILKVQEIRGYDQVTRIANTPAFIKGVTNLRGVIVPIVDLRVKFCEGDVEYDDNTVVIVLNLGQRVVGIVVDGVSDVLSLTAEQIRPAPEFAVTLSTEYLTGLGALGERMLILVNIEKLLNSEEMALLDIAASHVA

SEQ ID NO: 15 - protein, 671 aa, Salmonella typhimurium
MSMDISDFYQTFFDEADELLADMEQHLLDLVPESPDAEQLNAIFRAAHSIKGGAGTFGFTILQETTHLMENLLDEARRGEMQLNTDIINLFLETKDIMQEQLDAYKNSEEPDAASFEYICNALRQLALEAKGETTPAVVETAALSAAIQEESVAETESPRDESKLRIVLSRLKANEVDLLEEELGNLATLTDVVKGADSLSATLDGSVAEDDIVAVLCFVIEADQIAFEKVVAAPVEKAQEKTEVAPVAPPAVVAPAAKSAAHEHHAGREKPARERESTSIRVAVEKVDQLINLVGELVITQSMLAQRSNELDPVNHGDLITSMGQLQRNARDLQESVMSIRMMPMEYVFSRFPRLVRDLAGKLGKQVELTLVGSSTELDKSLIERIIDPLTHLVRNSLDHGIEMPEKRLEAGKNVVGNLILSAEHQGGNICIEVTDDGAGLNRERILAKAMSQGMAVNENMTDDEVGMLIFAPGFSTAEQVTDVSGRGVGMDVVKRNIQEMGGHVEIQSKQGSGTTIRILLPLTLAILDGMSVRVAGEVFILPLNAVMESLQPREEDLHPLAGGERVLEVRGEYLPLVELWKVFDVDGAKTEATQGIVVILQSAGRRYALLVDQLIGQHQVVVKNLESNYRKVPGISAATILGDGSVALIVDVSALQGLNREQRMAITAA

SEQ ID NO: 16 - protein, 239 aa, Salmonella typhimurium
MNSLYTAEGVMDKHSLWQRYVPLVRHEALRLQVRLPASVELDDLLQAGGIGLLNAVDRYDALQGTAFTTYAVQRIRGAMLDELRSRDWVPRSVRRNAREVAQAMGQLEQELGRNATETEVAERLGIPVAEYRQMLLDTNNSQLFSYDEWREEHGDSIELVTEEHQQENPLHQLLEGDLRQRVMDAIESLPEREQLVLTLYYQEELNLKEIGAVLEVGESRVSQLHSQAIKRLRTKLGKL

SEQ ID NO: 17 - protein, 266 aa, Salmonella typhimurium
MKLALLGRQALMGVMAVALVAGMSAKSFADEGLLNKVKERGTLLVGLEGTYPPFSFQGEDGKLTGFEVDFAEALAKHLGVKASLKPTKWDGMLASLDAKRIDVVINQVTISDVRKKKYDFSTPYTVSGIQALVKKGNEGTIKTAADLQGKKVGVGLGTNYEEWLRQHVQGVDIRTYDDDPTKYQDLRVGRIDAILVDRLAALDLVKKTKGTLAVTGDAFSRQESGVALRKGNEDLLKAVDNAIAEMQKDGTLKALSEKWFGADVTQ

SEQ ID NO: 18 - protein, 183 aa, Salmonella typhimurium
MTVQQPKRRPLSRYLKDFKHSQTHCAHCHKLLDRITLVRRGKIVNKIAISQLDMLLDDAAWQREQKEWVALCRFCGDLHCKKQSDFFDIIGFKQYLFEQTEMSHGTVREYVVRLRRLGNYLSEQNISHDLLQDGFLDESLAPWLPETSTNNYRIALRKYQQYKAHQQIAPRQKSPFTASSDIY

SEQ ID NO: 19 - protein, 506 aa, Salmonella typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 20 - protein, 135 aa, Salmonella typhimurium
MYTASGIKAYAQVSVESAVMSASPHQLIEMLFDGANSALVRARLFLEQGDVVAKGEALSKAINIIDNGLKAGLDQEKGGEIATNLSELYDYMIRRLLQANLRNDAQAIEEVEGLLSNIAEAWKQISPKASFQESR

SEQ ID NO: 21 - protein, 104 aa, Salmonella typhimurium
MAAIQGIEGVISQLQATAMAARGQDTHSQSTVSFAGQLHAALDRISDRQAAARVQAEKFTLGEPGIALNDVMADMQKASVSMQMGIQVRNKLVAAYQEVMSMQV

SEQ ID NO: 22 - protein, 560 aa, Salmonella typhimurium
MSATASTATQPKPLEWLNRLRANPRIPLIVAGSAAVAIVVAMVLWAKTPDYRTLFSNLSDQDGGAIVAQLTQMNIPYRFANGSGAIEVPADKVHELRLRLAQQGLPKGGAVGFELLDQEKFGISQFSEQVNYQRALEGELARTIETLGPVKSARVHLAMPKPSLFVREQKSPSASVTVTLEPGRALDEGQISAVVHLVSSAVAGLPPGNVTLVDQSGHLLTQSNTSGRDLNDAQLKFANDVESRIQRRIEAILSPIVGNGNVHAQVTAQLDFANKEQTEEHYSPNGDASKATLRSRQLNISEQVGAGYPGGVPGALSNQPAPPNEAPIATPPTNQQNAQNTPQTSTSTNSNSAGPRSTQRNETSNYEVDRTIRHTKMNVGDIERLSVAVVVNYKTLADGKPLPLTADQMKQIEDLTREAMGFSDKRGDTLNVVNSPFSAVDNTGGELPFWQQQSFIDQLLAAGRWLLVLVVAWILWRKAVRPQLTRRVEEAKAAQEQAQVRQETEEAVEVRLSKDEQLQQRRANQRLGAEVMSQRIREMSDNDPRVVALVIRQWMSNDHE

SEQ ID NO: 23 - protein, 147 aa, Salmonella typhimurium
MAQHGALETLKDLAEKEVDDAARLLGEMRRGCQQAEEQLKMLIDYQNEYRSNLNTDMGNGIASNRWINYQQFIQTLEKAIEQHRLQLTQWTQKVDLALKSWREKKQRLQAWQTLQDRQTAAALLAENRMDQKKMDEFAQRAAMRKPE

SEQ ID NO: 24 - protein, 155 aa, Salmonella typhimurium
MTDSAINKKSKRSIWIPLLVLITLAACATAGYSYWRMQQQPTTNAKAEPAPPPAPVFFALDTFTVNLGDADRVLYIGVTLRLKDEATRARLNEYLPEVRSRLLLLFSRQNAAELSTEAGKQKLIAAIKETLAAPLVAGQPKQVVTDVLYTAFILR

SEQ ID NO: 25 - protein, 334 aa, Salmonella typhimurium
MGDSILSQAEIDALLNGDSDTKDEPTPGIASDSDIRPYDPNTQRRVVRERLQALEIINERFARQFRMGLFNLLRRSPDITVGAIRIQPYHEFARNLPVPTNLNLIHLKPLRGTGLVVFSPSLVFIAVDNLFGGDGRFPTKVEGREFTHTEQRVINRMLKLALEGYSDAWKAINPLEVEYVRSEMQVKFTNITTSPNDIVVNTPFHVEIGNLTGEFNICLPFSMIEPLRELLVNPPLENSRHEDQNWRDNLVRQVQHSELELVANFADIPLRLSQILKLKPGDVLPIEKPDRIIAHVDGVPVLTSQYGTVNGQYALRVEHLINPILNSLNEEQPK

SEQ ID NO: 26 - protein, 137 aa, Salmonella typhimurium
MSDMNNPSDENTGALDDLWADALNEQKATTTKSAADAVFQQLGGGDVSGAMQDIDLIMDIPVKLTVELGRTRMTIKELLRLTQGSVVALDGLAGEPLDILINGYLIAQGEVVVVADKYGVRITDIITPSERMRRLSR

SEQ ID NO: 27 - protein, 125 aa, Salmonella typhimurium
MMKTEATVSQPTAPAGSPLMQVSGALIGIIALILAAAWVIKRMGFAPKGNSVRGLKVSASASLGPRERVVIVEVENARLVLGVTASQINLLHTLPPAENDTEAPVAPPADFQNMMKSLLKRSGRS

SEQ ID NO: 28 - protein, 245 aa, Salmonella typhimurium
MRRLLFLSLAGLWLFSPAAAAQLPGLISQPLAGGGQSWSLSVQTLVFITSLTFLPAILLMMTSFTRIIIVFGLLRNALGTPSAPPNQVLLGLALFLTFFIMSPVIDKIYVDAYQPFSEQKISMQEALDKGAQPLRAFMLRQTREADLALFARLANSGPLQGPEAVPMRILLPAYVTSELKTAFQIGFTIFIPFLIIDLVIASVLMALGMMMVPPATIALPFKLMLFVLVDGWQLLMGSLAQSFYS

SEQ ID NO: 29 - protein, 89 aa, Salmonella typhimurium
MTPESVMMMGTEAMKVALALAAPLLLVALITGLIISILQAATQINEMTLSFIPKIVAVFIAIIVAGPWMLNLLLDYVRTLFSNLPYIIG

SEQ ID NO: 30 - protein, 264 aa, Salmonella typhimurium
MIQVTSEQWLYWLHLYFWPLLRVLALISTAPILSERAIPKRVKLGLGIMITLVIAPSLPANDTPLFSIAALWLAMQQILIGIALGFTMQFAFAAVRTAGEFIGLQMGLSFATFVDPGSHLNMPVLARIMDMLAMLLFLTFNGHLWLISLLVDTFHTLPIGSNPVNSNAFMALARAGGLIFLNGLMLALPVITLLLTLNLALGLLNRMAPQLSIFVIGFPLTLTVGIMLMAALMPLIAPFCEHLFSEIFNLLADIVSEMPINNNP

SEQ ID NO: 31 - protein, 331 aa, Salmonella typhimurium
MSNLSGTDKSVILLMTIGEDRAAEVFKHLSTREVQALSTAMANVRQISNKQLTDVLSEFEQEAEQFAALNINANEYLRSVLVKALGEERASSLLEDILETRDTTSGIETLNFMEPQSAADLIRDEHPQIIATILVHLKRSQAADILALFDERLRHDVMLRIATFGGVQPAALAELTEVLNGLLDGQNLKRSKMGGVRTAAEIINLMKTQQEEAVITAVREFDGELAQKIIDEMFLFENLVDVDDRSIQRLLQEVDSESLLIALKGAEPPLREKFLRNMSQRAADILRDDLANRGPVRLSQVENEQKAILLIVRRLAETGEMVIGSGEDTYV

SEQ ID NO: 32 - protein, 235 aa, Salmonella typhimurium
MSNELPWQVWTPDDLAPPPETFVPVEADNVTLTEDTPEPELTAEQQLEQELAQLKIQAHEQGYNAGLAEGRQKGHAQGYQEGLAQGLEQGQAQAQTQQAPIHARMQQLVSEFQNTLDALDSVIASRLMQMALEAARQVIGQTPAVDNSALIKQIQQLLQQEPLFSGKPQLRVHPDDLQRVEEMLGATLSLHGWRLRGDPTLHHGGCKVSADEGDLDASVATRWQELCRLAAPGVL

SEQ ID NO: 33 - protein, 456 aa, Salmonella typhimurium
MTTRLTRWLTALDNFEAKMALLPAVRRYGRLTRATGLVLEATGLQLPLGATCIIERQDGPETKEVESEVVGFNGQRLFLMPLEEVEGILPGARVYARNGHGDGLQSGKQLPLGPALLGRVLDGGGKPLDGLPAPDTLETGALITPPFNPLQRTPIEHVLDTGVRAINALLTVGRGQRMGLFAGSGVGKSVLLGMMARYTRADVIVVGLIGERGREVKDFIENILGPDGRARSVVIAAPADVSPLLRMQGAAYATRIAEDFRDRGQHVLLIMDSLTRYAMAQREIALAIGEPPATKGYPPSVFAKLPALVERAGNGIHGGGSITAFYTVLTEGDDQQDPIADSARAILDGHIVLSRRLAEAGHYPAIDIEASISRAMTALITEQHYARVRLFKQLLSSFQRNRDLVSVGAYAKGSDPMLDKAITLWPQLEAFLQQGIFERADWEDSLQALDLIFPTV

SEQ ID NO: 34 - protein, 122 aa, Salmonella typhimurium
MTSTVEFINRWQRIALLSQSLLELAQRGEWDLLLQQEVSYLQSIETVMEKQTPPGITRSIQDMVAGYIKQTLDNEQLLKGLLQQRLDELSSLIGQSTRQKSLNNAYGRLSGMLLVPDAPGAS

SEQ ID NO: 35 - protein, 467 aa, Salmonella typhimurium
MASISSLGVGSNLPLDQLLTDLTKNEKGRLTPITKQQSANSAKLTAYGTLKSALEKFQTANTALNKADLFKSTVASSTTEDLKVSTTAGAAAGTYKINVTQLAAAQSLATKTTFATTKEQLGDTSVTSRTIKIEQPGRKEPLEIKLDKGDTSMEAIRDAINDADSGIAASIVKVKENEFQLVLTANSGTDNTMKITVEGDTKLNDLLAYDSTTNTGNMQELVKAENAKLNVNGIDIERQSNTVTDAPQGITLTLTKKVTDATVTVTKDDTKAKEAIKSWVDAYNSLVDTFSSLTKYTAVEPGEEASDKNGALLGDSVVRTIQTGIRAQFANSGSNSAFKTMAEIGITQDGTSGKLKIDDDKLTKVLKDNTAAARELLVGDGKETGITTKIATEVKSYLADDGIIDNAQDNVNATLKSLTKQYLSVSNSIDETVARYKAQFTQLDTMMSKLNNTSSYLTQQFTAMNKS

SEQ ID NO: 36 - protein, 495 aa, Salmonella typhimurium [sequence omitted]

SEQ ID NO: 37 - protein, 506 aa, Salmonella typhimurium; identical to SEQ ID NO: 19

SEQ ID NO: 38 - protein, 140 aa, Salmonella typhimurium
MTRLSEILDQMTTVLNDLKTVMDAEQQQLSVGQINGSQLQRITEEKSSLLATLDYLEQQRRLEQNAQRSANDDIAERWQAITEKTQHLRDLNQHNGWLLEGQIERNQQALEVLKPHQEPTLYGADGQTSVSHRGGKKISI

SEQ ID NO: 39 - protein, 97 aa, Salmonella typhimurium
MSIDRTSPLKPVSTVQTRETSDTPVQKTRQEKTSAATSASVTLSDAQAKLMQPGVSDINMERVEALKTAIRNGELKMDTGKIADSLIREAQSYLQSK

SEQ ID NO: 40 - protein, 219 aa, Salmonella typhimurium
MQTLKRGFAVAALLFSPLTMAQDINAQLTTWFSQRLAGFSDEVVVTLRSSPNLLPSCEQPAFSMTGSAKLWGNVNVVARCANEKRYLQVNVQATGNYVAVAAPIARGGKLTPANVTLKRGRLDQLPPRTVLDIRQIQDAVSLRDLAPGQPVQLTMIRQAWRVKAGQRVQVIANGEGFSVNAEGQAMNNAAVAQNARVRMTSGQIVSGTVDSDGNILINL

SEQ ID NO: 41 - protein, 138 aa, Salmonella typhimurium
MLDRLDAALRFQQEALNLRAQRQEILAANIANADTPGYQARDIDFASELKKVMVRGREETGGVALTLTSSHHIPAQAVSSPAVDLLYRVPDQPSLDGNTVDMDRERTQFADNSLKYQMGLTVLGSQLKGMMNVLQGGN

SEQ ID NO: 42 - protein, 134 aa, Salmonella typhimurium
MALLNIFDIAGSALAAQSKRLNVAASNLANADSVTGPDGQPYRAKQVVFQVDAAPGQATGGVKVASVIESQAPEKLVYEPGNPLADANGYVKMPNVDVVGEMVNTMSASRSYQANIEVLNTVKSMMLKTLTLGQ

SEQ ID NO: 43 - protein, 232 aa, Salmonella typhimurium
MSIAVNMNDPTNTGVKTTTGSGSMTGSNAADLQSSFLTLLVAQLKNQDPTNPLQNNELTTQLAQISTVSGIEKLNTTLGAISGQIDNSQSLQATTLIGHGVMVPGTTILAGKGAEEGAVTSTTPFGVELQQPADKVTATITDKDGRVVRTLEIGELRAGVHTFTWDGKQTDGTTVPNGSYNIAITASNGGTQLVAQPLQFALVQGVTKGSNGNLLDLGTYGTTTLDEVRQII

SEQ ID NO: 44 - protein, 403 aa, Salmonella typhimurium
MSFSQAVSGLNAAATNLDVIGNNIANSATYGFKSGTASFADMFAGSKVGLGVKVAGITQDFTDGTTTNTGRGLDVAISQNGFFRLVDSNGSVFYSRNGQFKLDENRNLVNMQGMQLTGYPATGTPPTIQQGANPAPITIPNTLMAAKSTTTASMQINLNSTDPVPSKTPFSVSDADSYNKKGTVTVYDSQGNAHDMNVYFVKTKDNEWAVYTHDSSDPAATAPTTASTTLKFNENGILESGGTVNITTGTINGATAATFSLSFLNSMQQNTGANNIVATNQNGYKPGDLVSYQINNDGTVVGNYSNEQEQVLGQIVLANFANNEGLASQGDNVWAATQASGVALLGTAGSGNFGKLTNGALEASNVDLSKELVNMIVAQRNYQSNAQTIKTQDQILNTLVNLR

SEQ ID NO: 45 - protein, 251 aa, Salmonella typhimurium
MDHAIYTAMGAASQTLNQQAVTASNLANASTPGFRAQLNALRAVPVDGLSLATRTLVTASTPGADMTPGQLDYTSRPLDVALQQDGWLVVQAADGAEGYTRNGNIQVGPTGQLTIQGHPVIGEGGPITVPEGSEITIAADGTISALNPGDPPNTVAPVGRLKLVKAEGNEVQRSDDGLFRLTAEAQAERGAVLAADPSIRIMSGVLEGSNVKPVEAMTDMIANARRFEMQMKVITSVDENEGRANQLLSMS

SEQ ID NO: 46 - protein, 260 aa, Salmonella typhimurium
MISSLWIAKTGLDAQQTNMDVIANNLANVSTNGFKRQRAVFEDLLYQTIRQPGAQSSEQTTLPSGLQIGTGVRPVATERLHSQGNLSQTNNSKDVAIKGQGFFQVMLPDGTSAYTRDGSFQVDQNGQLVTAGGFQVQPAITIPANALSITIGRDGVVSVTQQGQAAPVQVGQLNLTTFMNDTGLESIGENLYIETQSSGAPNESTPGLNGAGLLYQGYVETSNVNVAEELVNMIQVQRAYEINSKAVSTTDQMLQKLTQL

SEQ ID NO: 47 - protein, 232 aa, Salmonella typhimurium
MQKYALHAYPVMALMVATLTGCAWIPAKPLVQGATTAQPIPGPVPVANGSIFQSAQPINYGYQPLFEDRRPRNIGDTLTIVLQENVSASKSSSANASRDGKTSFGFDTVPRYLQGLFGNSRADMEASGGNSFNGKGGANASNTFSGTLTVTVDQVLANGNLHVVGEKQIAINQGTEFIRFSGVVNPRTISGSNSVPSTQVADARIEYVGNGYINEAQNMGWLQRFFLNLSPM

SEQ ID NO: 48 - protein, 365 aa, Salmonella typhimurium
MFKALAGIVLALVATLAHAERIRDLTSVQGVRENSLIGYGLVVGLDGTGDQTTQTPFTTQTLNNMLSQLGITVPTGTNMQLKNVAAVMVTASYPPFARQGQTIDVVVSSMGNAKSLRGGTLLMTPLKGVDSQVYALAQGNILVGGAGASAGGSSVQVNQLNGGRITNGAIIERELPTQFGAGNTINLQLNDEDFTMAQQITDAINRARGYGSATALDARTVQVRVPSGNSSQVRFLADIQNMEVNVTPQDAKVVINSRTGSVVMNREVTLDSCAVAQGNLSVTVNRQLNVNQPNTPFGGGQTVVTPQTQIDLRQSGGSLQSVRSSANLNSVVRALNALGATPMDLMSILQSMQSAGCLRAKLEII

SEQ ID NO: 49 - protein, 316 aa, Salmonella typhimurium
MIGDGKLLASAAWDAQSLNELKAKAGQDPAANIRPVARQVEGMFVQMMLKSMREALPKDGLFSSDQTRLYTSMYDQQIAQQMTAGKGLGLADMMVKQMTSGQTMPADDAPQVPLKFSLETVNSYQNQALTQLVRKAIPKTPDSSDAPLSGDSKDFLARLSLPARLASEQSGVPHHLILAQAALESGWGQRQILRENGEPSYNVFGVKATASWKGPVTEITTTEYENGEAKKVKAKFRVYSSYLEALSDYVALLTRNPRYAAVTTAATAEQGAVALQNAGYATDPNYARKLTSMIQQLKAMSEKVSKTYSANLDNLF

SEQ ID NO: 50 - protein, 553 aa, Salmonella typhimurium
MSSLINHAMSGLNAAQAALNTVSNNINNYNVAGYTRQTTILAQANSTLGAGGWIGNGVYVSGVQREYDAFITNQLRGAQNQSSGLTTRYEQMSKIDNLLADKSSSLSGSLQSFFTSLQTLVSNAEDPAARQALIGKAEGLVNQFKTTDQYLRDQDKQVNIAIGSSVAQINNYAKQIANLNDQISRMTGVGAGASPNDLLDQRDQLVSELNKIVGVEVSVQDGGTYNLTMANGYTLVQGSTARQLAAVPSSADPTRTTVAYVDEAAGNIEIPEKLLNTGSLGGLLTFRSQDLDQTRNTLGQLALAFADAFNAQHTKGYDADGNKGKDFFSIGSPVVYSNSNNADKTVSLTAKVVDSTKVQATDYKIVFDGTDWQVTRTADNTTFTATKDADGKLEIDGLKVTVGTGAQKNDSFLLKPVSNAIVDMNVKVTNEAEIAMASESKLDPDVDTGDSDNRNGQALLDLQNSNVVGGNKTFNDAYATLVSDVGNKTSTLKTSSTTQANVVKQLYKQQQSVSGVNLDEEYGNLQRYQQYYLANAQVLQTANALFDALLNIR

SEQ ID NO: 51 - protein, 317 aa, Salmonella typhimurium
MRISTQMMYEQNMSGITNSQAEWMKLGEQMSTGKRVTNPSDDPIAASQAVVLSQAQAQNSQYALARTFATQKVSLEESVLSQVTTAIQTAQEKIVYAGNGTLSDDDRASLATDLQGIRDQLMNLANSTDGNGRYIFAGYKTEAAPFDQATGGYHGGEKSVTQQVDSARTMVIGHTGAQIFNSITSNAVPEPDGSDSEKNLFVMLDTAIAALKTPVEGNNVEKEKAAAAIDKTNRGLKNSLNNVLTVRAELGTQLSELSTLDSLGSDRALGQKLQMSNLVDVDWNSVISSYVMQQAALQASYKTFTDMQGMSLFQLNR

SEQ ID NO: 52 - DNA, 531 bp, Acinetobacter sp.
  source 1..531: mol_type = other DNA
ccagctgatcagttgtagaatgaaaaaataagtgcctgccagaatcaaaaaagttccagccaaaaatagatattgcatcaaaccttggaatgagctcaattcaatcagtcgttcataaaaacgagcatttgtaaaacttgctaaccaacaagctagaagcccattaaatgtatttatagataaacttacttttttgaattgtagcaatttaaacatctcaatatcgttcagttatttgaggggtatagctacattaaagagtatgaaaattaagagaaaattagttttcgcttcaatgattcaatttaaatctaaatatcaatgttttaagatcataatgatggatatgaaaacttaaacctataccggataggcaattaagaaagaaatatgatataattgttttaagatattgattttaataaattatttaaaaaattaataactgctttaaaaaactttaaattggatttaattgagtttgcagactaaaaataaaacaataaatcaagttctcaaaggaaatgagtc

SEQ ID NO: 53 - protein, 11 aa, unidentified
AANDENYALAA

SEQ ID NO: 54 - protein, 11 aa, unidentified
AANDENYADAS

SEQ ID NO: 55 - protein, 13 aa, unidentified
AANDENYNYADAS

SEQ ID NO: 56 - DNA, 891 bp, unidentified
  source 1..891: mol_type = other DNA
atggacctgtctctgatccgtatcttcatctgcgtttacgaaaacaaaaacatctctaaagcggcggaaatcctgaacctgtctcagccgtctgttacctacaacctgaaccgtctgcgtaaacacctgaacaacccgctgttcgaacgtacccagtacggtgttgaagcgaccaaactgtctcacgaactgtacccggttttcaaagaatctatcctgaaaatcgaaatcgcggttgacgaagcgctgaacttcaacccgctgacctctaacaaaaccttccgtatcggtctgtctgacatcggtgaaatctgcctgctgccgaccctgatcgaatacctgcgtgcgcacgcgccgaaaatcaaaatcgaagttgaagaaatcaaaatcgaccaggttgaaaaatggctgatcgaaggtttcatcgacgttgcggttttcaactctacccacctggaattcaaacacctggaatacgaaaccctgttcctggaacgttacgttgcgctggttaacatgaaccacccgcgtatccgttctaccctgtctttcgacgcgtacctgaacgaatctcacgttgcgatcaaatcttctaccggtcacacccaggttgaccacgttctgaaactgatgggtcaccagcgtaaaatcgcgctggaagttccgcacttcggtgttctgcagggtgttctggacaaaaccgacctgatggttaccctgccgtctcgtgcggcgcagcagtacctgaaccagtctcacgttcgtgttctggaactgccgttccagatgtctgaattctacgttggtctgcactggttcgcgcagaccaacgaaccgctggcgcgtatctggctgatccagacctgcaaaaaagttatctctgttctgtaa

SEQ ID NO: 57 - protein, 296 aa, unidentified
MDLSLIRIFICVYENKNISKAAEILNLSQPSVTYNLNRLRKHLNNPLFERTQYGVEATKLSHELYPVFKESILKIEIAVDEALNFNPLTSNKTFRIGLSDIGEICLLPTLIEYLRAHAPKIKIEVEEIKIDQVEKWLIEGFIDVAVFNSTHLEFKHLEYETLFLERYVALVNMNHPRIRSTLSFDAYLNESHVAIKSSTGHTQVDHVLKLMGHQRKIALEVPHFGVLQGVLDKTDLMVTLPSRAAQQYLNQSHVRVLELPFQMSEFYVGLHWFAQTNEPLARIWLIQTCKKVISVL

SEQ ID NO: 58 - DNA, 1023 bp, unidentified
  source 1..1023: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccggcacgctacaggacgcttttgacctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaaaacctttgtgggcggtaactgtaccgttagcctgatgttgatgtcgctgggcggtctgtttgcccataatctcgttgactgggtatccgttgcaacttatcaagcagcttctggtggtggtgcacgtcatatgcgtgagctgttaacccagatgggtcagttgtatggccatgtcgccgatgaactggcgacgccgagctccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttc

SEQ ID NO: 59 - protein, 390 aa, unidentified
MVKDAPQDTGAHTQHISLQEKNAMKNVGFIGWRGMVGSVLMQRMVEERDFDAIRPVFFSTSQFGQAAPTFGDTSGTLQDAFDLDALKALDIIVTCQGGDYTNEIYPKLRESGWQGYWIDAASTLRMKDDAIIILDPVNQDVITDGLNNGVKTFVGGNCTVSLMLMSLGGLFAHNLVDWVSVATYQAASGGGARHMRELLTQMGQLYGHVADELATPSSAILDIERKVTALTRSGELPVDNFGVPLAGSLIPWIDKQLDNGQSREEWKGQAETNKILNTASVIPVDGLCVRVGALRCHSQAFTIKLKKEVSIPTVEELLAAHNPWAKVVPNDRDITMRELTPAAVTGTLTTPVGRLRKLNMGPEFLSAFTVGDQLLWGAAEPLRRMLRQLA

SEQ ID NO: 60 - DNA, 526 bp, unidentified
  source 1..526: mol_type = other DNA
atggattataaagatgatgatgataaaatgaaatataccagctatattctggcgtttcctgtgcattgtgctgggcagcctgggctgctattgccaggatccgtatgtgaaagaagcggaaaacctgaaaaaatattttaacgcgggccatagcgatgtggcggataacggcaccctgtttctgggcattctgaaaaactggaaagaagaaagcgatcgcaaaattatgcagagccagattgtgagcttttattttaaactgtttaaaaactttaaagatgatcagagcattcagaaaagcgtggaaaccattaaagaagatatgaacgtgaaattttttaacagcaacaaaaaaaaacgcgatgattttgaaaaactgaccaactatagcgtgaccgatctgaacgtgcagcgcaaagcgattcatgaactgattcaggtgatggcggaactgagcccggcggcgaaaaccggcaaacgcaaacgcagccagatgctgtttcgcggccgccgcgcgagccagtaa

SEQ ID NO: 61 - protein, 175 aa, Homo sapiens
MDYKDDDDKMKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ